(19) United States
(12) Patent Application Publication
(10) Pub. No.: US 2025/0354999 A1
(43) Pub. Date: Nov. 20, 2025

(54) BIOMARKERS OF LYSOSOMAL STORAGE DISEASE

(71) Applicant: Genzyme Corporation, Cambridge, MA (US)

(72) Inventors: Matthew Douglas Davison, Cambridge, MA (US); Matthew Christopher Koehler, Cambridge, MA (US); Hongge Wang, Cambridge, MA (US); Katherine Wood Klinger, Cambridge, MA (US); Tai-He Xia, Cambridge, MA (US); Bailin Zhang, Cambridge, MA (US)

(21) Appl. No.: 18/995,596

(22) PCT Filed: Jul. 18, 2023
(86) PCT No.: PCT/IB2023/057323

§ 371 Date: Jan. 16, 2025

Related U.S. Application Data
(60) Provisional application No. 63/390,377, filed on Jul. 19, 2022.
(60) Provisional application No. 63/444,659, filed on Feb. 10, 2023.

(30) Foreign Application Priority Data
Oct. 14, 2022 (EP) 22201741.0

(51) Int. Cl. G01N 33/68

(52) U.S. Cl. CPC G01N 33/6872; G01N 33/6893; G01N 2333/70596; G01N 2800/04; G01N 2800/52

(57) ABSTRACT

Provided are methods for assessing lysosomal dysfunction, as well as for diagnosing specific lysosomal storage diseases. The methods employ CD63 as a biomarker and can be used for the diagnosis and monitoring of conditions, as well as for the monitoring and/or adjustment of therapeutic interventions for such conditions. Also provided are assays and kits for use in said methods.

### CROSS-REFERENCE TO RELATED APPLICATIONS

This application is a national phase entry pursuant to 35 U.S.C. § 371 of International Application No. PCT/IB2023/057323, filed Jul. 18, 2023, which claims the benefit to U.S. Provisional Application No. 63/390,377, filed Jul. 19, 2022, European Application No. 22201741.0, filed Oct. 14, 2022, and U.S. Provisional Application No. 63/444,659, filed Feb. 10, 2023, the entire contents of each of which are incorporated by reference herein in their entirety for any purpose.

This disclosure relates to biomarkers of lysosomal storage disease. The disclosure provides methods useful in the diagnosis and monitoring of specific lysosomal storage diseases, as well as for the monitoring and/or adjustment of therapeutic interventions for such conditions.

### SUMMARY

Lysosomal storage disorders (LSDs) are a group of genetic diseases which include Fabry disease (FD), Gaucher disease (GD), and Mucopolysaccharidoses (MPS). These disorders mostly involve the dysfunction of lysosomal hydrolases, which results in impaired substrate degradation. Disruption of lysosomal function can lead to the accumulation of undegraded substrate(s) in endosomes and lysosomes, eventually compromising cellular function. Although each LSD typically results from a mutation in a different gene, with a consequent deficiency of enzyme activity or protein function, all LSDs share a common biochemical characteristic in that they result in an accumulation of substrates within lysosomes.

Although lysosomal proteins are ubiquitously distributed, the accumulation of undegraded substrate(s) in LSD patients is normally restricted to those cells, tissues, and organs in which substrate turnover is high. The accumulation of the primary storage material can cause a chain of secondary disruptions to other biochemical and cellular functions, which leads to the severe pathology in lysosomal storage disorders.

The extent and severity of a LSD typically depends on the type and amount of substrate that accumulates, but almost all of the disorders are progressive. Many clinical similarities are observed between groups as well as within each group. Common clinical features of many LSDs include bone abnormalities, organomegaly, central nervous system dysfunction, and coarse hair and facial features. Many patients with lysosomal storage disorders die in infancy or childhood, and patients who survive to adulthood often have a decreased lifespan and significant morbidity.

Individual LSDs are classified as rare diseases, but their prevalence is significant when considered as a group of disorders and they represent an important health issue. Limited numbers of studies have investigated the incidence of LSDs, defined as the total number of cases diagnosed within a certain period, divided by the total number of live births in the same period. One of the main problems associated with obtaining accurate epidemiological data for these individually rare disorders is that, in most countries, there are numerous diagnostic centers which compounds the problem of collecting and correlating diagnoses. The combined estimated prevalence of LSDs worldwide is around 1 in 7,500 live births. The true prevalence is likely greater due to misdiagnosed or undiagnosed cases.

Gaucher disease (GD) is an inherited metabolic disorder caused by mutations in the GBA gene which result in deficiency of glucocerebrosidase (GCase). Accumulation of the primary storage material, glucosylceramide (GL1), in the lysosomes of macrophages affects cells of the reticuloendothelial system, including liver, spleen, and bone marrow. It is the most common LSD with a prevalence of roughly 1 in 40,000 in the general population. In the Ashkenazi Jewish population, the prevalence was historically as high as 1 in 1,000.

Fabry disease (FD) is the second most common of the LSDs, after Gaucher disease. It is an X-linked lysosomal storage disorder characterized by deficient activity of the enzyme alpha-galactosidase A (α-Gal) encoded by the GLA gene. Enzyme deficiency results in the progressive intracellular accumulation of glycosphingolipids, mostly globotriaosylceramide (GL3), in a variety of cell types and tissues including kidney, heart, liver, spleen, and skin, as well as in the peripheral and central nervous systems.

Mucopolysaccharidoses (collectively “MPS”) are inherited autosomal recessive disorders (except for MPS type II, which is X-linked) where mucopolysaccharides—also known as glycosaminoglycans (GAGs)—accumulate in connective and other tissues throughout the body such as skin, cartilage, cornea, liver, spleen, and vascular tissue. Severe presentation is considered a pre-school age child with developmental delay, short stature, recurrent ear and respiratory infections, hepatosplenomegaly, and coarsening of facial features. Over time, the child develops hearing loss, cardiac valve disease, airway obstruction, skeletal contractures, and distinctive facial appearance with macrocephaly, thick eyebrows, gingival hypertrophy, macroglossia, and thickening of the lips and nasal alae. Intellect is impaired, and patients undergo regression as the disease progresses. IQ is <70 in 61% of patients who are untreated and borderline in 25% at 2-3 years. Other complications include corneal clouding, carpal tunnel syndrome, hydrocephalus, glaucoma, cardiac arrhythmias, cervical instability, and spinal cord compression. Untreated, the life expectancy is the second or third decade.

Currently, there is no cure for any LSD, and there are no approved treatments for many of these conditions. A particular challenge lies in developing effective therapies for the treatment of CNS manifestations, which are common in LSDs. For the LSDs where treatments are available, quality of life has significantly improved for these patients, especially those whose disease was diagnosed and treated at an early stage.

Existing treatments for LSDs, where these are available, typically involve either enzyme replacement therapy (ERT), in which an active version of the deficient enzyme is administered to the patient to compensate for the reduced or aberrant activity of the patient's own enzyme, or substrate reduction therapy (SRT), in which a modulator of an enzyme involved in the deficient lysosomal pathway is administered to change the flux of substrates through the pathway and reduce levels of the problematic substrates. In either case, regular and repeated administration of the therapy is required. About 70% of approved therapies involve ERT. Authorised treatments for FD are based on ERT using recombinant α-Gal, namely agalsidase beta (Fabrazyme®—Sanofi, EU approval 2001, US approval 2003) and agalsidase alfa (Replagal®—Takeda, EU approval 2001). FD patients can also be treated with a small molecule chaperone, migalastat (Galafold®—Amicus Therapeutics, EU approval 2016), if they have an amenable GLA mutation. Treatments for GD include ERT, e.g. imiglucerase (Cerezyme®—Sanofi, EU approval 1997), and SRT, e.g. eliglustat (Cerdelga®—Sanofi, EU approval 2015) and miglustat (Zavesca®—Janssen, EU approval 2002). Treatment for MPS may involve ERT using alpha-L-iduronidase (Aldurazyme®—Sanofi, EU approval 2003 for MPS I). Other active agents are being evaluated for the treatment of lysosomal storage diseases; one such agent is venglustat, i.e. (3S)-1-azabicyclo[2.2.2]octan-3-yl N-{2-[2-(4-fluorophenyl)-1,3-thiazol-4-yl]propan-2-yl}carbamate, which is in phase 2 clinical trials as a SRT treatment for FD.

Before treatment of a LSD can begin, it is necessary to diagnose the condition. Given the relative rarity of LSDs, a method of diagnosis should aim for a high level of sensitivity and specificity. Historically, a diagnosis would be made based on multiple factors including family history, clinical manifestations, and biopsies, although this could miss early (pre-symptomatic) disease; this is especially problematic in the case of severe childhood LSDs. More recently, genetic screening and enzyme or substrate assays have been developed to investigate some LSDs. Such tests are, however, typically highly specific to each LSD or even to sub-types of each LSD, and there is no broad diagnostic assay for the group of LSDs in general which utilises a single biomarker. Multiplex screening assays have been proposed but these have yet to be widely adopted (see, e.g., international patent publication WO 2004/088322 which measures levels of multiple lysosomal protein markers such as LAMP-1, saposin C, α-glucosidase and α-iduronidase in neonate blood spots).

A canonical biomarker for FD, which was used up until the early 2000's, was globotriaosylceramide (GL3)—the major accumulating substrate resulting from α-Gal inactivity. A growing uncertainty about the role of GL3 in disease pathology, as well as a lack of correlation between GL3 and α-Gal activity or between GL3 and disease manifestations, raised the need for a more robust diagnostic and prognostic biomarker. α-Gal activity itself was considered to have diagnostic potential in male patients, but its diagnostic sensitivity in female patients is poor. In the late 2000's, the deacylated form of GL3, globotriaosylsphingosine (lyso-GL3), was investigated as a biomarker for FD and was found to have superior sensitivity compared to GL3 and α-Gal activity. Lyso-GL3 is now an important diagnostic biomarker and is often used to supplement full gene analysis in determining cases of FD. It is also useful for monitoring disease progression and treatment.

A similar rationale was used to develop diagnostic tests using biomarkers for GD. This disease is characterised by the accumulation of glucosylceramide (GL1), and its deacylated form glucosylsphingosine (lyso-GL1), in the body because of a deficiency in GCase. Lyso-GL1 levels, β-glucosidase activity, and genetic GBA sequencing are currently the most common diagnostic and prognostic indicators for GD. In the case of lyso-GL1, some studies suggest that its pathological involvement is correlated with disease burden and clinical severity. The proteins chitotriosidase and CCL18 have also been identified as biomarkers for GD but are not commonly used in the clinic.

Mucopolysaccharidosis type I (MPS I), also known as Hurler syndrome, is characterized by a deficiency in the alpha-L-iduronidase (IDUA) enzyme, which results in the accumulation of its catabolic substrates, dermatan sulfate and heparan sulfate, in lysosomes. Most often in clinical settings, the first tier of MPS I diagnosis is IDUA enzyme activity. If enzyme activity is decreased, then a second-tier test of mucopolysaccharidosis is performed on blood or blood spots to look for elevated dermatan sulfate and heparan sulfate. Along with diagnosis, dermatan sulfate and heparan sulfate levels are the standard for treatment monitoring. There are similar assays, looking at the levels of specific GAGs, which can be used to diagnose and/or monitor other mucopolysaccharidoses.

For many LSDs, however, there are no clinically validated biomarkers for diagnosis or for monitoring treatment progression. There is, therefore, an acute need to develop biomarkers for early detection of LSDs in general, as well as a need to develop new and improved methods for characterising and monitoring specific LSDs.

The present application demonstrates that the cell-surface glycoprotein CD63 can be used as a biomarker to diagnose and/or monitor multiple LSDs. Thus, CD63 can act as a common biomarker for LSD pathogenesis. Not only does CD63 function as a biomarker for disease progression on its own, but measurement of CD63 levels can also be used in conjunction with other clinical measures to diagnose and/or monitor specific conditions.

CD63 is a protein which was first detected as a marker of platelet activation, although its precise function is unknown. It localizes to the membranes of melanosomes and platelet dense bodies. Some cells are enriched in CD63, such as activated basophils and proliferating mast cells, and CD63 is often used in cell biology as a marker for multivesicular bodies. It is also used as a marker for extracellular vesicles released from either the multivesicular body or the plasma membrane. CD63 can also be used as a cell marker, e.g., to quantify platelet size, number, or volume (see, e.g., WO 2004/088322, above). CD63 is heavily glycosylated, which may protect it from lysosomal enzyme degradation. When the structural gene and cDNA for CD63 were first isolated and sequenced, it was found to be identical to ME491—an antigen associated with early melanoma cells. CD63 also appears to be identical to granulophysin, a protein associated with platelet dense bodies. Because of its known uses, there are many commercial kits available for its detection and quantification in biological samples. These are typically based on an ELISA using a specific anti-CD63 antibody.

Based on the observations described in the present examples, although without wishing to be bound by theory, it is postulated that CD63 can act as a circulating biomarker for multiple lysosomal storage diseases, especially those that share the same glycosphingolipid pathway. Moreover, because the level of CD63 in a patient can be more stable over time than the levels of conventional biomarkers for LSDs (e.g., for GD), CD63 represents a particularly advantageous biomarker for monitoring specific LSDs.

Accordingly, a first aspect provides a method of diagnosing a subject as suffering from, or being at risk of suffering from, a lysosomal storage disease (LSD), the method comprising measuring the level of CD63 in a sample from the subject, wherein CD63 is the only biomarker which is employed in the method.

In embodiments, the subject is diagnosed as suffering from, or being at risk of suffering from, a lysosomal storage disease which includes Fabry disease, Gaucher disease, MPS type I, MPS type II, and MPS type III.

Another aspect provides a method of detecting or diagnosing lysosomal dysfunction in a subject, the method comprising measuring the level of CD63 in a sample from the subject, wherein CD63 is the only biomarker which is employed in the method.

Another aspect provides a method of detecting or diagnosing aberrant glycosphingolipid processing in a subject, the method comprising measuring the level of CD63 in a sample from the subject, wherein CD63 is the only biomarker which is employed in the method.

Another aspect provides a method for generating quantitative data for a subject, the method comprising determining the level of a single biomarker in a sample from the subject, wherein the biomarker is CD63.

In embodiments, the subject has not previously been diagnosed with a LSD and/or has not been assessed for risk factors associated with lysosomal dysfunction or aberrant glycosphingolipid processing. In embodiments, the sample comprises (e.g. consists of) a blood fraction selected from plasma and serum.

In embodiments, the subject is diagnosed as suffering from, or being at risk of suffering from, a lysosomal storage disease, or is diagnosed as having lysosomal dysfunction or aberrant glycosphingolipid processing, if the level of CD63 is greater than a control value, wherein the control value is measured as the CD63 level in a sample taken either from the same subject at an earlier point in time or from one or more healthy subjects.

Another aspect provides the use of CD63 as a biomarker in the diagnosis of a lysosomal storage disease in a subject, the detection or diagnosis of lysosomal dysfunction in a subject, or the detection or diagnosis of aberrant glycosphingolipid processing in a subject, wherein CD63 is used as the only biomarker in said detection or diagnosis.

Another aspect provides a method of diagnosing Fabry disease in a subject suspected as being at risk of suffering from Fabry disease, the method comprising measuring the level of CD63 in a sample from the subject.

In embodiments, the subject is suspected as being at risk of suffering from Fabry disease as a result of presenting with one or more of the following: family history of Fabry disease, fatigue, pain, lenticular or corneal opacity, vortex keratopathy, angiokeratoma, shortness of breath, palpitations, edema, renal disease, myocardial dysfunction, conduction abnormalities with reduced PR-interval, cardiac arrhythmias, vertigo, headache, diplopia, dysarthria, hemiataxia, transient ischemic attacks, premature stroke, and dementia. In embodiments, the subject is diagnosed with Fabry disease if the level of CD63 measured in the sample from the subject is greater than a control value, wherein the control value is measured as the CD63 level in a sample taken from one or more healthy subjects. In embodiments, the subject is diagnosed with Fabry disease if the level of CD63 measured in the sample from the subject is at least about 100% greater than the control value.

Another aspect provides a method for generating quantitative data for a subject, wherein the method comprises (e.g. consists of) determining the level of CD63 in a sample from the subject, wherein the subject is suffering from Fabry disease or is suspected of suffering from Fabry disease.

Another aspect provides the use of CD63 as a biomarker in the diagnosis of Fabry disease in a subject suspected as being at risk of suffering from Fabry disease.

Another aspect provides the use of CD63 as a biomarker to improve a method of diagnosing Fabry disease in a subject, optionally wherein CD63 is used as a biomarker alongside GL3, lyso-GL3, and/or α-Gal activity.

Another aspect provides a method of treating a subject who has been diagnosed as having Fabry disease by a method as defined hereinbefore, the treatment comprising administering to the subject one or more therapeutic treatments for Fabry disease.

Another aspect provides a method of treating Fabry disease in a patient in need thereof, wherein the patient has a higher than normal blood plasma CD63 level, the method comprising administering to the subject an effective amount of a therapeutic treatment for Fabry disease.

Another aspect provides a method for generating quantitative data for a subject, wherein the method comprises: (a) determining a level of CD63 in a sample from the subject; (b) determining whether the level of CD63 is greater than a control value, wherein the control value is measured as the CD63 level in a sample taken from one or more healthy subjects; and (c) applying one or more therapeutic treatments for Fabry disease to the subject if the level of CD63 in the sample is greater than the control value.

In embodiments, the one or more therapeutic treatments comprise (e.g. consist of) substrate reduction therapy, chaperone therapy, enzyme replacement therapy, and/or gene therapy. In embodiments, the treatment comprises administering venglustat or migalastat to the subject, e.g. venglustat. In other embodiments, the treatment comprises administering recombinant α-galactosidase to the subject, e.g. agalsidase beta.

Another aspect provides a therapeutic agent for the treatment of Fabry disease in a subject, wherein the subject has been diagnosed as having Fabry disease by a method as defined hereinbefore.

In embodiments, the one or more therapeutic treatments comprise (e.g. consist of) substrate reduction therapy, chaperone therapy, enzyme replacement therapy, and/or gene therapy. In embodiments, the treatment comprises administering venglustat or migalastat to the subject, e.g. venglustat. In other embodiments, the treatment comprises administering recombinant α-galactosidase to the subject, e.g. agalsidase beta.

Another aspect provides a method for monitoring the progress of Fabry disease in a subject diagnosed as having Fabry disease, the method comprising: (a) measuring the level of CD63 in a first sample from the subject; (b) measuring the level of CD63 in a second sample from the subject, wherein the second sample was taken from the subject after the first sample was taken from the subject; (c) comparing the level of CD63 in the first sample with the level of CD63 in the second sample; and (d) determining that the Fabry disease in the subject is becoming more severe if the level of CD63 in the second sample is greater than the level of CD63 in the first sample, determining that the Fabry disease in the subject is not progressing if the level of CD63 in the second sample is substantially the same as the level of CD63 in the first sample, and determining that the Fabry disease in the subject is becoming less severe if the level of CD63 in the second sample is lower than the level of CD63 in the first sample.

Another aspect provides a method for generating quantitative data for a subject diagnosed as having Fabry disease, the method comprising the steps of: (a) determining a level of CD63 in a first sample from the subject; (b) determining a level of CD63 in a subsequent sample from the subject; and (c) comparing the level of CD63 determined in step (a) with the level determined in step (b).

In embodiments, the sample is a blood sample, e.g. a plasma sample.

Another aspect provides a method for monitoring the progress of a treatment for Fabry disease in a subject diagnosed as having Fabry disease, the method comprising: (a) measuring the level of CD63 in a first sample from the subject; (b) administering a therapeutic treatment for Fabry disease to the subject; (c) measuring the level of CD63 in a second sample from the subject, wherein the second sample is taken from the subject after the therapeutic treatment was administered; and (d) determining that the treatment is successful if the level of CD63 in the second sample is lower than the level of CD63 in the first sample.

Another aspect provides a method of treating or preventing the development or progression of Fabry disease in a subject assessed as being at risk of suffering from Fabry disease, the method comprising the steps of: (a) taking a first biological sample from the subject and analyzing said sample for CD63 concentration; (b) if the CD63 concentration is above a control value, starting the subject on a course of therapeutic treatment for Fabry disease, and optionally: (c) after treating the subject, taking a second biological sample from the subject and analyzing said sample for CD63 concentration to determine a change in CD63 level; and (d) adjusting the therapeutic treatment based on the change in CD63 level observed.

Another aspect provides a method for adjusting the dosage of a therapeutic treatment for Fabry disease in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample.

Another aspect provides a method for generating quantitative data for a subject having Fabry disease, the method comprising the steps of: (a) determining a level of CD63 in a first sample from the subject; (b) determining a level of CD63 in a second sample from the subject after administration of a therapeutic treatment for Fabry disease to the subject.

In embodiments, the dosage of the therapeutic treatment is to be increased if the level of CD63 determined in step (b) is substantially the same as, or greater than, the level of CD63 determined in step (a).

In embodiments, the therapeutic treatment comprises (e.g. consists of) substrate reduction therapy, chaperone therapy, enzyme replacement therapy, or gene therapy. In embodiments, the treatment comprises administering venglustat or migalastat to the subject, e.g. venglustat. In other embodiments, the treatment comprises administering recombinant α-galactosidase to the subject, e.g. agalsidase beta.

In embodiments, the second or subsequent sample is taken from the subject at least 8 weeks after initiation of the therapeutic treatment.

Another aspect provides the use of CD63 as a biomarker for monitoring the progress of Fabry disease in a subject diagnosed as having Fabry disease, or for monitoring the progress of a treatment for Fabry disease, or for adjusting the dosage of a therapeutic treatment for Fabry disease, in a subject diagnosed as having Fabry disease.

Another aspect provides a method of diagnosing Gaucher disease in a subject suspected as being at risk of suffering from Gaucher disease, the method comprising measuring the level of CD63 in a sample from the subject.

In embodiments, the subject is suspected as being at risk of suffering from Gaucher disease as a result of presenting with one or more of the following: family history of Gaucher disease, hepatomegaly and splenomegaly, pain, osteoporosis, skin pigmentation, pancytopenia, neurological symptoms, and parkinsonism. In embodiments, the subject is diagnosed with Gaucher disease if the level of CD63 measured in the sample from the subject is greater than a control value, wherein the control value is measured as the CD63 level in a sample taken from one or more healthy subjects. In embodiments, the subject is diagnosed with Gaucher disease if the level of CD63 measured in the sample from the subject is at least about 100% greater than the control value.

Another aspect provides a method for generating quantitative data for a subject, wherein the method comprises (e.g. consists of) determining the level of CD63 in a sample from the subject, wherein the subject is suffering from Gaucher disease or is suspected of suffering from Gaucher disease.

Another aspect provides the use of CD63 as a biomarker in the diagnosis of Gaucher disease in a subject suspected as being at risk of suffering from Gaucher disease.

Another aspect provides the use of CD63 as a biomarker to improve a method of diagnosing Gaucher disease in a subject, optionally wherein CD63 is used as a biomarker alongside GL1, lyso-GL1, and/or β-glucosidase (GCase) activity.

Another aspect provides a method of treating a subject who has been diagnosed as having Gaucher disease by a method as defined hereinbefore, the treatment comprising administering to the subject one or more therapeutic treatments for Gaucher disease.

Another aspect provides a method of treating Gaucher disease in a patient in need thereof, wherein the patient has a higher than normal blood plasma CD63 level, the method comprising administering to the subject an effective amount of a therapeutic treatment for Gaucher disease.

Another aspect provides a method for generating quantitative data for a subject, wherein the method comprises: (a) determining a level of CD63 in a sample from the subject; (b) determining whether the level of CD63 is greater than a control value, wherein the control value is measured as the CD63 level in a sample taken from one or more healthy subjects; and (c) applying one or more therapeutic treatments for Gaucher disease to the subject if the level of CD63 in the sample is greater than the control value.

In embodiments, the one or more therapeutic treatments comprise (e.g. consist of) substrate reduction therapy, chaperone therapy, enzyme replacement therapy, and/or gene therapy. In embodiments, the treatment comprises administering venglustat, eliglustat, or miglustat to the subject. In embodiments, the treatment comprises administering recombinant glucocerebrosidase to the subject, e.g. imiglucerase.

Another aspect provides a therapeutic agent for the treatment of Gaucher disease in a subject, wherein the subject has been diagnosed as having Gaucher disease by a method as defined hereinbefore.

In embodiments, the therapeutic agent is a therapeutic treatment as defined hereinbefore.

Another aspect provides a method for monitoring the progress of Gaucher disease in a subject diagnosed as having Gaucher disease, the method comprising: (a) measuring the level of CD63 in a first sample from the subject; (b) measuring the level of CD63 in a second sample from the subject, wherein the second sample was taken from the subject after the first sample was taken from the subject; (c) comparing the level of CD63 in the first sample with the level of CD63 in the second sample; and (d) determining that the Gaucher disease in the subject is becoming more severe if the level of CD63 in the second sample is greater than the level of CD63 in the first sample, determining that the Gaucher disease in the subject is not progressing if the level of CD63 in the second sample is substantially the same as the level of CD63 in the first sample, and determining that the Gaucher disease in the subject is becoming less severe if the level of CD63 in the second sample is lower than the level of CD63 in the first sample.

Another aspect provides a method for generating quantitative data for a subject diagnosed as having Gaucher disease, the method comprising the steps of: (a) determining a level of CD63 in a first sample from the subject; (b) determining a level of CD63 in a subsequent sample from the subject; and (c) comparing the level of CD63 determined in step (a) with the level determined in step (b).

In embodiments, the sample is a blood sample, e.g. a plasma sample.

Another aspect provides a method for monitoring the progress of a treatment for Gaucher disease in a subject diagnosed as having Gaucher disease, the method comprising: (a) measuring the level of CD63 in a first sample from the subject; (b) administering a therapeutic treatment for Gaucher disease to the subject; (c) measuring the level of CD63 in a second sample from the subject, wherein the second sample is taken from the subject after the therapeutic treatment was administered; and (d) determining that the treatment is successful if the level of CD63 in the second sample is lower than the level of CD63 in the first sample.

Another aspect provides a method of treating or preventing the development or progression of Gaucher disease in a subject assessed as being at risk of suffering from Gaucher disease, the method comprising the steps of: (a) taking a first biological sample from the subject and analyzing said sample for CD63 concentration; (b) if the CD63 concentration is above a control value, starting the subject on a course of therapeutic treatment for Gaucher disease, and optionally: (c) after treating the subject, taking a second biological sample from the subject and analyzing said sample for CD63 concentration to determine a change in CD63 level; and (d) adjusting the therapeutic treatment based on the change in CD63 level observed.

Another aspect provides a method for adjusting the dosage of a therapeutic treatment for Gaucher disease in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample.

Another aspect provides a method for generating quantitative data for a subject having Gaucher disease, the method comprising the steps of: (a) determining a level of CD63 in a first sample from the subject; (b) determining a level of CD63 in a second sample from the subject after administration of a therapeutic treatment for Gaucher disease to the subject.

In embodiments, the dosage of the therapeutic treatment is to be increased if the level of CD63 determined in step (b) is substantially the same as, or greater than, the level of CD63 determined in step (a). In embodiments, the therapeutic treatment comprises (e.g. consists of) substrate reduction therapy, chaperone therapy, enzyme replacement therapy, or gene therapy. In embodiments, the treatment comprises administering venglustat, eliglustat, or miglustat to the subject. In embodiments, the treatment comprises administering recombinant glucocerebrosidase to the subject, e.g. imiglucerase. In embodiments, the second or subsequent sample is taken from the subject at least 8 weeks after initiation of the therapeutic treatment.

Another aspect provides the use of CD63 as a biomarker for monitoring the progress of Gaucher disease in a subject diagnosed as having Gaucher disease, or for monitoring the progress of a treatment for Gaucher disease, or for adjusting the dosage of a therapeutic treatment for Gaucher disease, in a subject diagnosed as having Gaucher disease.

Another aspect provides a method of diagnosing MPS in a subject suspected as being at risk of suffering from MPS, the method comprising measuring the level of CD63 in a sample from the subject.

In embodiments, the subject is suspected as being at risk of suffering from MPS as a result of presenting with one or more of the following: family history of MPS, macrocephaly, hearing loss, corneal clouding, abnormal dentition, stiffness, hip dysplasia, claw hands, joint laxity, valve thickening, left ventricular hypertrophy, recurrent respiratory infections, obstructive airway disease, hepatomegaly/splenomegaly, umbilical/inguinal hernia, developmental delay, ventriculomegaly, dilated perivascular spaces, hyperactive or aggressive behaviour, abnormal granulation in leukocytes, fetal hydrops, and proteinuria. In embodiments, the subject is diagnosed with MPS if the level of CD63 measured in the sample from the subject is greater than a control value, wherein the control value is measured as the CD63 level in a sample taken from one or more healthy subjects. In embodiments, the subject is diagnosed with MPS if the level of CD63 measured in the sample from the subject is at least about 100% greater than the control value.

Another aspect provides a method for generating quantitative data for a subject, wherein the method comprises (e.g. consists of) determining the level of CD63 in a sample from the subject, wherein the subject is suffering from MPS or is suspected of suffering from MPS.

Another aspect provides the use of CD63 as a biomarker in the diagnosis of MPS in a subject suspected as being at risk of suffering from MPS.

Another aspect provides the use of CD63 as a biomarker to improve a method of diagnosing MPS in a subject, optionally wherein CD63 is used as a biomarker alongside one or more glycosaminoglycans (GAGs) or glycans.

Another aspect provides a method of treating a subject who has been diagnosed as having MPS by a method as defined hereinbefore, the treatment comprising administering to the subject one or more therapeutic treatments for MPS.

Another aspect provides a method of treating MPS in a patient in need thereof, wherein the patient has a higher than normal blood plasma CD63 level, the method comprising administering to the subject an effective amount of a therapeutic treatment for MPS.

Another aspect provides a method for generating quantitative data for a subject, wherein the method comprises: (a) determining a level of CD63 in a sample from the subject; (b) determining whether the level of CD63 is greater than a control value, wherein the control value is measured as the CD63 level in a sample taken from one or more healthy subjects; and (c) applying one or more therapeutic treatments for MPS to the subject if the level of CD63 in the sample is greater than the control value.

In embodiments, the one or more therapeutic treatments comprise (e.g. consist of) enzyme replacement therapy, gene therapy, and/or hematopoietic stem cell transplantation. In embodiments, the treatment comprises (e.g. consists of) enzyme replacement therapy with α-L-iduronidase, iduronidase-2-sulfatase, or heparan-N-sulfatase.

Another aspect provides a therapeutic agent for the treatment of MPS in a subject, wherein the subject has been diagnosed as having MPS by a method as defined hereinbefore.

In embodiments, the therapeutic agent is a therapeutic treatment as defined hereinbefore.

Another aspect provides a method for monitoring the progress of MPS in a subject diagnosed as having MPS, the method comprising: (a) measuring the level of CD63 in a first sample from the subject; (b) measuring the level of CD63 in a second sample from the subject, wherein the second sample was taken from the subject after the first sample was taken from the subject; (c) comparing the level of CD63 in the first sample with the level of CD63 in the second sample; and (d) determining that the MPS in the subject is becoming more severe if the level of CD63 in the second sample is greater than the level of CD63 in the first sample, determining that the MPS in the subject is not progressing if the level of CD63 in the second sample is substantially the same as the level of CD63 in the first sample, and determining that the MPS in the subject is becoming less severe if the level of CD63 in the second sample is lower than the level of CD63 in the first sample.

Another aspect provides a method for generating quantitative data for a subject diagnosed as having MPS, the method comprising the steps of: (a) determining a level of CD63 in a first sample from the subject; (b) determining a level of CD63 in a subsequent sample from the subject; and (c) comparing the level of CD63 determined in step (a) with the level determined in step (b).

In embodiments, the sample is a blood sample, e.g. a plasma sample.

Another aspect provides a method for monitoring the progress of a treatment for MPS in a subject diagnosed as having MPS, the method comprising: (a) measuring the level of CD63 in a first sample from the subject; (b) administering a therapeutic treatment for MPS to the subject; (c) measuring the level of CD63 in a second sample from the subject, wherein the second sample is taken from the subject after the therapeutic treatment was administered; and (d) determining that the treatment is successful if the level of CD63 in the second sample is lower than the level of CD63 in the first sample.

Another aspect provides a method of treating or preventing the development or progression of MPS in a subject assessed as being at risk of suffering from MPS, the method comprising the steps of: (a) taking a first biological sample from the subject and analyzing said sample for CD63 concentration; (b) if the CD63 concentration is above a control value, starting the subject on a course of therapeutic treatment for MPS, and optionally: (c) after treating the subject, taking a second biological sample from the subject and analyzing said sample for CD63 concentration to determine a change in CD63 level; and (d) adjusting the therapeutic treatment based on the change in CD63 level observed.

Another aspect provides a method for adjusting the dosage of a therapeutic treatment for MPS in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample.

Another aspect provides a method for generating quantitative data for a subject having MPS, the method comprising the steps of: (a) determining a level of CD63 in a first sample from the subject; (b) determining a level of CD63 in a second sample from the subject after administration of a therapeutic treatment for MPS to the subject.

In embodiments, the dosage of the therapeutic treatment is to be increased if the level of CD63 determined in step (b) is substantially the same as, or greater than, the level of CD63 determined in step (a). In embodiments, the therapeutic treatment comprises (e.g. consists of) enzyme replacement therapy, gene therapy, and/or hematopoietic stem cell transplantation. In embodiments, the treatment comprises (e.g. consists of) enzyme replacement therapy with α-L-iduronidase, iduronidase-2-sulfatase, or heparan-N-sulfatase. In embodiments, the second or subsequent sample is taken from the subject at least 8 weeks after initiation of the therapeutic treatment.

Another aspect provides the use of CD63 as a biomarker for monitoring the progress of MPS in a subject diagnosed as having MPS, or for monitoring the progress of a treatment for MPS, or for adjusting the dosage of a therapeutic treatment for MPS, in a subject diagnosed as having MPS.

Another aspect provides a kit for detecting or diagnosing a specific lysosomal storage disease in a subject, the kit comprising: (a) means for detecting CD63 in a sample from the subject; and (b) means for detecting one or more biomarkers of said lysosomal storage disease in a sample from the subject.

In embodiments, the specific lysosomal storage disease is Fabry disease, and the kit comprises: (a) means for detecting CD63 in a sample from the subject; and (b) means for detecting one or more biomarkers of Fabry disease in the sample (e.g., means for detecting GL3 and/or lyso-GL3 in the sample).

In other embodiments, the specific lysosomal storage disease is Gaucher disease, and the kit comprises: (a) means for detecting CD63 in a sample from the subject; and (b) means for detecting one or more biomarkers of Gaucher disease in the sample (e.g., means for detecting lyso-GL1 in the sample).

In yet other embodiments, the lysosomal storage disease is MPS and the kit comprises: (a) means for detecting CD63 in a sample from the subject; and (b) means for detecting one or more biomarkers of MPS in the sample. In embodiments: (i) the lysosomal storage disease is MPS I and the kit comprises in part (b) means for detecting dermatan sulfate and optionally also heparan sulfate in the sample; (ii) the lysosomal storage disease is MPS II and the kit comprises in part (b) means for detecting dermatan sulfate and heparan sulfate in the sample; or (iii) the lysosomal storage disease is MPS III and the kit comprises in part (b) means for detecting heparan sulfate in the sample.

In embodiments, the means for detecting CD63 comprises at least one anti-CD63 antibody.

Additional features and advantages of compounds, compositions, and methods disclosed herein will be apparent from the following detailed description.

### BRIEF DESCRIPTION OF THE DRAWINGS

FIGS. 1A-1C shows the response of biomarkers to venglustat treatment in clinical trials over time in Fabry disease patients as compared to healthy controls. The biomarkers analysed are: CD63 (FIG. 1A)—labelled as “linear NPX”, i.e. normalized protein expression; GL3 (FIG. 1B); and lyso-GL3 (FIG. 1C). All biomarkers show a response to treatment. CD63 and lyso-GL3 do not return to control levels during the trial period. GL3 has no separation between baseline and control.

FIGS. 2A-2C shows the estimated marginal mean (log 2) values for CD63 (FIG. 2A), GL3 (FIG. 2B), and lyso-GL3 (FIG. 2C), shown longitudinally from baseline to 156 weeks on venglustat treatment for Fabry disease. Horizontal lines above the curve denote statistically significant differences, and the stars denote the confidence of the significant differences (*: p<0.05, **: p<0.01, ***: p<0.001, ****: p<0.0001). The first significant decrease in CD63 occurs by week 26.

FIGS. 3A-3C shows intraclass correlation coefficient (ICC) data (10,000 permutations) for biomarkers measured from the Fabry ACT/LTS Venglustat phase II clinical trial. ICC (FIG. 3A) is a measure of variability between patients, whereas ICC residual (FIG. 3C) is a measure of variability within patients. ICC distribution intercept is shown in FIG. 3B. Each plot shows, left to right: log 2 (CD63); log 2 (GL3); and log 2 (lyso-GL3). CD63 and lyso-GL3 have comparable within-subject variability, but both are much lower than GL3.

FIG. 4 shows a probability density function comparing the distribution of CD63 values (log 2) for healthy subjects (left) and diseased, treated Gaucher patients (right); sample metadata is listed in Table 2 below. There is a statistically significant separation between the two groups, with CD63 being upregulated in disease patients (Wilcoxon test: p=8.89e⁻¹⁰).

FIGS. 5A-5C shows pairwise spaghetti plots to visualize within-patient variability of CD63 (FIG. 5A), lyso-GL1 (LGL1; FIG. 5B), and chitotriosidase (CHITO; FIG. 5C) in Gaucher patients. The within-subject variability is visualized by plotting biomarker change between adjacent timepoints, i.e. the change as compared with the previous measurement. All patients included in the plot have been receiving treatment for Gaucher disease for several years, representing an on-treatment biomarker steady state. Results for each patient are shown on a separate line (and in a different line style). Note that data were not collected for all patients at each timepoint. Lyso-GL1 and CHITO appear more variable than CD63, and this is confirmed in FIG. 6.

FIGS. 6A-6D shows correlations between CD63 and other biomarkers. FIG. 6A to FIG. 6C show intraclass correlation coefficient (ICC) data (10,000 permutations) for biomarkers measured from the Gaucher cohort outlined in Table 2 below. ICC (FIG. 6A) is a measure of variability between patients, whereas ICC residual (FIG. 6C) is a measure of variability within patients. ICC distribution intercept is shown in FIG. 6B. Each plot shows, left to right: log 2 (CD63); log 2 (lyso-GL1); and log 2 (CHITO). Note that CD63 has significantly lower within-patient variability than either lyso-GL1 or CHITO. FIG. 6D presents the correlation in another way: the data points show the levels of CD63 in the samples from Gaucher patients versus lyso-GL1 level (light grey circles), CCL18 level (black triangles), and chitotriosidase activity (dark grey squares). Lines of best-fit are shown, with lyso-GL1 (lower line, light grey) having r=0.65 and p<0.0001; CCL18 (middle line, black) having r=0.63 and p=0.0119, and chitotriosidase (upper line, dark grey) having r=0.78 and p<0.0001.

FIGS. 7A-7B shows a probability density plot comparing the distribution of CD63 values (log 2) for healthy subjects and diseased MPS patients (FIG. 7A); all sample metadata is listed in Table 3 below. The shaded area to the right of the plot contains data from all 3 MPS sub-types. The curves within the shaded area to the right represent the distributions of the different MPS sub-types (shown with arrows). Statistical differences between the healthy and MPS distributions are shown in FIG. 8C. Also shown is a comparison of CD63 values between male and female MPS disease subjects (FIG. 7B). At α=0.05, the two distributions are not statistically different (p=0.0746).

FIGS. 8A-8C shows the results from the Fabry (FIG. 8A), Gaucher (FIG. 8B) and MPS (FIG. 8C) studies analysed separately for male and female patients (and controls). P-values are provided in each plot. The only instance of p>0.05 is for female Fabry patients.

FIG. 9 shows the Olink® proteome profiling of samples from male adult Fabry patients (baseline) as compared to healthy controls. Proteins with at least a 1.5-fold change and p<0.05 were considered significant and are shown in grey. CD63 is circled.

FIG. 10 shows a boxplot of CD63 levels in CSF samples from healthy control (left-most plot) as compared to MPS I (middle plot) and MPS II (right-most plot) patients.

FIG. 11 shows CD63 levels in CSF samples from healthy controls as compared to patients with type 3 Gaucher disease (GD3); GD3 samples were taken at three timepoints: baseline, 26 weeks, and 52 weeks on treatment with venglustat and imiglucerase. Average levels are elevated in GD3 patients at baseline as compared to healthy controls and reduce over time (FIG. 11A). This trend is also seen in individual patients (FIG. 11B).

### DETAILED DESCRIPTION

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology, and recombinant DNA, which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al., eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g. pH, temperature, time, concentration, molecular weight, etc., including ranges, are approximations which are varied (+) or (−) by increments of, e.g., 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, but do not exclude others.

“Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like.

“Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

A “subject,” “individual”, or “patient” is used interchangeably herein, and refers to a vertebrate, such as a mammal. Mammals include, but are not limited to, murines, rats, rabbit, simians, bovines, ovine, porcine, canines, felines, farm animals, sport animals, pets, equines, primates, and humans. In embodiments, the mammals include horses, dogs, and cats. In embodiments, the mammal is a human.

“Administering” is defined herein as a means of providing an agent or a composition containing the agent to a subject in a manner that results in the agent being contacted with (e.g. being inside) the subject's body. Such an administration can be by any route including, without limitation, oral, transdermal (e.g. vagina, rectum, or oral mucosa), by injection (e.g. subcutaneous, intravenous, parenterally, intraperitoneally, into the CNS), or by inhalation (e.g. oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms.

The term “suffering from” (or “having”) as it relates, in particular, to a lysosomal storage disease refers to a patient or individual who has developed at least some of the characteristic pathologies of the disease and/or is displaying one or more signs of the disease (e.g., one or more biomarkers characteristic of the disease), irrespective of whether or not they have actually been diagnosed with the disease. A patient may be referred to as being “at risk of suffering” from such a disease where they are predisposed to the disease (e.g., having a history of disease in their family lineage or because of the presence of genetic mutations associated with the disease) but where they have not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents for any particular subject depends upon a variety of factors including, for example, the activity of the specific compound employed, the age, body weight, general health, sex, and diet of the subject, the time of administration, the rate of excretion, the drug combination, and the severity of the particular disorder being treated, and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with a lysosomal storage disease. For example, oral administration may require a total daily dose of from 0.1 mg to 1000 mg of active agent. The total daily dose may be administered in single or divided doses and may, at the physician's discretion, fall outside of the typical range given herein.

Where reference is made herein to compounds, e.g. eliglustat or venglustat, these encompass the compound per se as well as the pharmaceutically acceptable salts thereof. For example, references to “eliglustat” include eliglustat hemitartrate, and references to “venglustat” include venglustat malate. Venglustat is(S)-1-azabicyclo[2.2.2]octan-3-yl-N-[2-[2-(4-fluorophenyl)-1,3-thiazol-4-yl]propan-2-yl]carbamate. Eliglustat is N-[(1R,2R)-1-(2,3-dihydro-1,4-benzodioxin-6-yl)-1-hydroxy-3-pyrrolidin-1-ylpropan-2-yl]octanamide.

Where appropriate, any embodiments (e.g, methods) provided herein can be combined with any one or more of the other embodiments (e.g, methods) provided herein.

The following abbreviations are used herein:
α-Gal alpha-galactosidase A
BCA bicinchoninic acid
BL baseline
BSA bovine serum albumen
CCL18 chemokine (C—C motif) ligand 18
CDNA complementary DNA
CHITO chitotriosidase
CNS central nervous system
CS chondroitin sulfate
CSF cerebrospinal fluid
CV confidence value
DNA deoxyribonucleic acid
DS dermatan sulfate
ELISA enzyme-linked immunosorbent assay
ERT enzyme replacement therapy
ESI electrospray ionisation
FACS fluorescence assisted cell sorting
FD Fabry disease
GAGs glycosaminoglycans
GBA glucocerebrosidase gene
GCase glucocerebrosidase (also called acid β-glucosidase)
GD Gaucher disease
GD3 type 3 Gaucher disease
GI gastrointestinal
GL1 glucosylceramide
GL3 globotriaosylceramide
GM1 monosialotetrahexosylganglioside
GM2 monosialotrihexosylganglioside
GM3 monosialodihexosylganglioside
HS heparan sulfate
HSCT hematopoietic stem cell transplantation
ICC intraclass correlation coefficient
IDUA alpha-L-iduronidase
IQ intelligence quotient
KS keratan sulfate
LCMS liquid chromatography mass spectrometry
LC-MS/MS liquid chromatography tandem mass spectrometry
LSD lysosomal storage disorder
lyso-GL1 glucosylsphingosine (also referred to as LGL1)
lyso-GL3 globotriaosylsphingosine
MPS mucopolysaccharidosis (or mucopolysaccharidoses)
NCL neuronal ceroid lipofuscinosis(es)
NPX normalized protein expression
PEA proximity extension assay
PBS phosphate buffered saline
PCR polymerase chain reaction
RNA ribonucleic acid
SD standard deviation
SEM standard error of the mean
SRT substrate reduction therapy
Tris tris(hydroxymethyl)aminomethane
UPLC ultra high performance liquid chromatography
WK week

### Methods of Diagnosing or Detecting Lysosomal Storage Diseases Generally

Provided herein is a method of diagnosing a subject as suffering from, or being at risk of suffering from, a lysosomal storage disease, the method comprising measuring the level of CD63 in a sample from the subject. This method has the advantage of being able to detect irregularities in the glycosphingolipid pathway which are common to many LSDs. Thus, the method is able to provide a general diagnosis of lysosomal storage disorders, i.e., the diagnosis does not have to be limited to the assessment of a single disease state but provides a more holistic picture of lysosomal function and glycosphingolipid processing. A related aspect therefore provides a method of detecting or diagnosing lysosomal dysfunction in a subject, the method comprising measuring the level of CD63 in a sample from the subject. Another related aspect provides a method of detecting or diagnosing aberrant glycosphingolipid processing in a subject, the method comprising measuring the level of CD63 in a sample from the subject. In embodiments, CD63 is the only biomarker which is employed in the above methods.

Other related aspects provide the use of CD63 as a biomarker in the diagnosis of a lysosomal storage disease in a subject. Related aspects provide the use of CD63 as a biomarker in the detection or diagnosis of lysosomal dysfunction in a subject. Related aspects provide the use of CD63 as a biomarker in the detection or diagnosis of aberrant glycosphingolipid processing in a subject. In embodiments, CD63 is used as the only biomarker in said detection and/or diagnosis.

Viewed in another way, these aspects provide a method for generating quantitative data for a subject, the method comprising determining the level of a biomarker in a sample from the subject, wherein the biomarker is CD63. In embodiments, the method comprises determining the level of a single biomarker, i.e., CD63.

While the subject to be assessed may have been identified as being at risk of suffering from a LSD or suspected of having a LSD, e.g., by considering family history or clinical observations, it is envisaged that the present method will be carried out on a subject whose risk factors for lysosomal disease or dysfunction have not already been assessed. Thus, in embodiments the subject has not previously been diagnosed with a LSD and/or has not been assessed for risk factors associated with lysosomal dysfunction or aberrant glycosphingolipid processing. Such risk factors include, but are not limited to, the subject having parents with genetic mutations known to cause LSDs; including parents of Ashkenazi Jewish, Finnish, Asian, or Dutch heritage; or parents who are related to each other.

As noted above, the present methods can provide a general diagnosis of lysosomal storage disorder. Without wishing to be bound by theory, it is postulated that the methods may be suitable to diagnose a large class of diseases including Fabry disease, Krabbe disease, Gaucher disease (e.g. type 1, 2, and 3), Niemann-Pick disease (e.g. type A, B, and C), metachromatic leukodystrophy, Farber disease, Krabbe disease, galactosialidosis, Schindler disease, GM1 gangliosidosis, GM2 gangliosidoses (e.g. AB variant, Sandhoff disease, and Tay-Sachs disease), GM3 gangliosidosis, Lysosomal acid lipase deficiency, Wolman disease, cholesteryl ester storage disease, multiple sulfatase deficiency, Pompe disease, Danon disease, Salla disease, alpha-mannosidosis, beta-mannosidosis, aspartylglucosaminuria, fucosidosis, MPS I (e.g. Hurler syndrome, Scheie syndrome, and Hurler-Scheie syndrome), MPS II (e.g. Hunter syndrome), MPS III (e.g. Sanfilippo syndrome type A, B, C, and D), MPS type IV (e.g. Morquio syndrome type A and B), MPS type VI (e.g. Maroteaux-Lamy syndrome), MPS type VII (e.g. Sly syndrome), MPS type IX (e.g. hyaluronidase deficiency), mucolipidosis (e.g. sialidosis, inclusion cell disease, pseudo-Hurler polydystrophy/phosphotransferase deficiency, and mucolipidin 1 deficiency), and neuronal ceroid lipofuscinoses (e.g. Santavuori-Haltia disease/infantile NCL, Jansky-Bielschowsky disease/late infantile NCL, Batten-Spielmeyer-Vogt disease/juvenile NCL, Kufs disease/adult NCL, Finnish variant/Type 5, Late infantile variant/type 6, type 7, Northern epilepsy/Turkish late infantile/type 8, German/Serbian late infantile/type 9, and Congenital cathepsin D deficiency). Thus, in embodiments the subject is diagnosed as suffering from, or being at risk of suffering from, a lysosomal storage disease selected from the aforementioned conditions. In other embodiments, the subject is diagnosed as suffering from, or being at risk of suffering from, a lysosomal storage disease selected from Fabry disease, Krabbe disease, Gaucher disease (e.g. type 1, 2, and 3), metachromatic leukodystrophy, Farber disease, Krabbe disease, galactosialidosis, Schindler disease, GM1 gangliosidosis, GM2 gangliosidoses (e.g. AB variant, Sandhoff disease, and Tay-Sachs disease), GM3 gangliosidosis, Lysosomal acid lipase deficiency, Wolman disease, cholesteryl ester storage disease, multiple sulfatase deficiency, Pompe disease (e.g. infantile-onset Pompe disease), Danon disease, Salla disease, alpha-mannosidosis, beta-mannosidosis, aspartylglucosaminuria, fucosidosis, MPS I (e.g. Hurler syndrome, Scheie syndrome, and Hurler-Scheie syndrome), MPS II (e.g. Hunter syndrome), MPS III (e.g. Sanfilippo syndrome type A, B, C, and D), MPS type IV (e.g. Morquio syndrome type A and B), MPS type VI (e.g. Maroteaux-Lamy syndrome), MPS type VII (e.g. Sly syndrome), MPS type IX (e.g. hyaluronidase deficiency), mucolipidosis (e.g. sialidosis, inclusion cell disease, pseudo-Hurler polydystrophy/phosphotransferase deficiency, and mucolipidin 1 deficiency), and neuronal ceroid lipofuscinoses (e.g. Santavuori-Haltia disease/infantile NCL, Jansky-Bielschowsky disease/late infantile NCL, Batten-Spielmeyer-Vogt disease/juvenile NCL, Kufs disease/adult NCL, Finnish variant/Type 5, Late infantile variant/type 6, type 7, Northern epilepsy/Turkish late infantile/type 8, German/Serbian late infantile/type 9, and Congenital cathepsin D deficiency). In yet other embodiments, the subject is diagnosed as suffering from, or being at risk of suffering from, a lysosomal storage disease selected from Fabry disease, Gaucher disease, MPS type I, MPS type II, and MPS type III. In embodiments, the Gaucher disease is Gaucher disease type 1, 2, or 3 (e.g., type 3).

The sample from the subject in which the CD63 is measured or determined can be any sample which contains lysosomes, exosomes, and/or other cell fractions in which CD63 may build up after impairment of lysosomal function. The sample may consist essentially of one sample type (e.g., one tissue or fluid type) or it may consist of a plurality of sample types (e.g., several tissue and/or fluid types). The sample type may, for example, be selected from blood, a blood fraction, urine, cerebrospinal fluid, sputum, lymph, dermal tissue, renal tissue, cardiac tissue, spleen tissue, bone marrow, and the like. Most conveniently, the sample is of a type which can be obtained in a relatively non-invasive manner. Thus, in embodiments the sample from the subject comprises (e.g., consists of) blood, a blood fraction, and/or urine. In particular embodiments, the sample from the subject comprises (e.g., consists of) a blood fraction selected from plasma and serum. In other embodiments, the sample from the subject comprises (e.g., consists of) CSF.

In making a diagnosis of a lysosomal storage disease or detecting lysosomal dysfunction or aberrant glycosphingolipid processing, the level of CD63 in the sample will typically be compared to a control value to determine whether or not it is within a normal (e.g. healthy) range or outside a normal range. Determining a normal range of values for any given sample type is within the capabilities of the skilled person. As described in detail herein, the detection and quantification of CD63 in a sample is typically carried out using an immunoassay such as an ELISA, although other methods may be employed (e.g., as described herein). There are multiple commercial sources for suitable ELISA kits, such as those mentioned below and in the Examples.

In embodiments, the method comprises measuring the level of CD63 in a sample from the subject (i.e. the test sample level) and comparing that level to a control value, wherein the test sample level is considered to be outside the normal range if it is greater than the control value. In one embodiment, the control value is a baseline CD63 level which has previously been measured in the same subject (i.e. in an earlier sample from the subject), for example measured at least 1 month prior to obtaining the test sample from the subject, e.g. at least 2, 3, 6, 9, 12, 18, or 24 months prior to obtaining the test sample from the subject. The control value may represent the CD63 level measured in a single earlier sample from the subject or it may be an average of values from multiple earlier samples. In an alternative embodiment, the control value is the CD63 level in a sample taken from a healthy subject (or an average value for a cohort of healthy subjects), i.e. a subject which does not suffer from and is not at risk of suffering from a lysosomal storage disease such as those as listed above. In embodiments, the healthy subjects are matched to the subject being assessed, e.g. by age and/or sex. In particular embodiments, the control value represents the level of CD63 in the same type of sample which is being assessed in the present methods, e.g., a sample which has been obtained, processed, and/or stored in the same way as the test sample. For example, where the present method utilizes a plasma sample, the control value typically represents the level of CD63 in a control plasma sample, e.g., an earlier plasma sample taken from the same subject or a plasma sample obtained from one or more healthy subjects.

In embodiments where the control value for CD63 concentration is a point value (or an average, e.g. mean, value), the CD63 level in the test sample is considered to be outside the normal range if it is numerically greater than the control value. For example, it may be at least about 5% greater than the control value, e.g. at least about 7.5%, 10%, 12.5%, 15%, 20%, 25%, 30%, 40%, 50%, 75%, or 100% greater than the control value. In particular, it may be at least about 125% greater than the control value, e.g. at least about 150%, 175%, 200%, 250%, 300%, 350%, 400%, or 500% greater than the control value. In embodiments, the CD63 level in the test sample is from about 200% to about 700% greater (e.g. from about 300% to about 500% greater, or from about 350% to about 450% greater) than the control value.

In embodiments where the control value for CD63 concentration is a range of values, e.g. described by some variability around a mean value, the CD63 level in the test sample is considered to be outside the normal range if it is more than about 1 standard error greater than the mean value, e.g. more than about 1.0, 1.1, 1.2, 1.3, 1.4, 1.5, 1.6, 1.7, 1.8, 1.9, 2.0, 2.5, or 3.0 standard errors greater than the mean value. In other embodiments (e.g. where the patient is female and/or is suspected to have or be at risk of suffering from Fabry disease), the CD63 level in the test sample is considered to be outside the normal range if it is more than about 0.25 standard errors greater than the mean value, e.g. more than about 0.3, 0.4, 0.5, 0.6, 0.7, 0.8, or 0.9 standard errors greater than the mean value.

Thus, in embodiments is provided a method of diagnosing a subject as suffering from, or being at risk of suffering from, a lysosomal storage disease, the method comprising:
measuring the level of CD63 in a test sample from the subject (e.g. a plasma sample);
comparing the level which is measured in the test sample to a control value, wherein the control value is measured as the CD63 level in a sample (e.g. a plasma sample) taken either from the same subject at an earlier point in time or from one or more healthy subjects; and
diagnosing the subject as suffering from, or being at risk of suffering from, a lysosomal storage disease if the level which is measured in the test sample is greater than the control value.

In other embodiments is provided a method of detecting or diagnosing lysosomal dysfunction in a subject, the method comprising:
measuring the level of CD63 in a test sample from the subject (e.g. a plasma sample);
comparing the level which is measured in the test sample to a control value, wherein the control value is measured as the CD63 level in a sample (e.g. a plasma sample) taken either from the same subject at an earlier point in time or from one or more healthy subjects; and
detecting or diagnosing lysosomal dysfunction in the subject if the level which is measured in the test sample is greater than the control value.

In other embodiments is provided a method of detecting or diagnosing aberrant glycosphingolipid processing in a subject, the method comprising:
measuring the level of CD63 in a test sample from the subject (e.g. a plasma sample);
comparing the level which is measured in the test sample to a control value, wherein the control value is measured as the CD63 level in a sample (e.g. a plasma sample) taken either from the same subject at an earlier point in time or from one or more healthy subjects; and
detecting or diagnosing aberrant glycosphingolipid processing in the subject if the level which is measured in the test sample is greater than the control value.

The above embodiments may optionally include a step of obtaining the test sample from the subject, e.g. taking a blood sample and optionally separating the blood sample into a fraction thereof (e.g. a plasma sample).

### Methods of Diagnosing, Treating, and Monitoring Specific Conditions

As well as being useful in the detection and diagnosis of lysosomal dysfunction and aberrant glycosphingolipid processing, as detailed above, CD63 can also be used as a biomarker in the diagnosis, monitoring, and therapeutic treatment of specific lysosomal storage diseases.

Thus, in one aspect is provided a method for the diagnosis of a specific lysosomal storage disease (e.g. a condition as defined herein) in a subject, the method comprising measuring the level of CD63 in a sample from the subject. A related aspect provides the use of CD63 as a biomarker in the diagnosis of a specific lysosomal storage disease in a subject. Viewed in another way, these aspects provide a method for generating quantitative data for a subject, who may be suspected as being at risk of suffering from a specific lysosomal storage disease, the method comprising determining the level of CD63 in a sample from the subject. A related aspect provides the use of CD63 as a biomarker to improve a method of diagnosing a specific lysosomal storage disease in a subject.

It will be appreciated that, where the present methods are directed towards specific conditions, these aspects will typically rely on additional measures besides the CD63 level in the test sample from the subject. For example, the methods may be performed on a subject who is already suspected as being at risk of suffering from the specific LSD, and/or they may employ other biomarkers which are characteristic for the condition in question. A subject may be considered as being at risk of suffering from the disease through the identification of specific risk factors such as family history of the disease, genetic testing, the analysis of other characteristic biomarkers, clinical symptoms, and the like. In this case, the CD63 level in the sample from the subject is typically measured after the risk factors are assessed, such that the subject is already considered to be at risk of suffering from the disease; here the CD63 measurement is acting to confirm a diagnosis of lysosomal dysfunction, and hence disease, in that subject. Alternatively, measurement of the CD63 level in the sample from the subject is used alongside one or more other characteristic measures of the disease, e.g., as part of a panel of biomarkers used for diagnostic purposes. In this case, the CD63 measurement serves to improve the accuracy of the diagnosis, e.g., to reduce the proportion of false positives and/or increase the proportion of true positives in a population of test subjects (as compared to a corresponding diagnostic method in which the CD63 level is not taken into account).

This diagnosis of a specific lysosomal storage disease in a subject can be used to direct a therapeutic treatment for the disease. Thus, in one aspect is provided a method of treating a subject who has been diagnosed as having a specific lysosomal storage disease by a method as defined hereinbefore, the treatment comprising administering to the subject one or more therapeutic treatments for said disease. A related aspect provides a therapeutic agent for the treatment of a specific lysosomal storage disease in a subject, wherein the subject has been diagnosed as having said disease by a method as defined hereinbefore. Also provided is a method for generating quantitative data for a subject, wherein the method comprises (e.g. consists of): (a) determining a level of CD63 in a sample from the subject; (b) determining whether the level of CD63 is greater than a control value (e.g. a control value measured as the CD63 level in a sample taken from one or more healthy subjects); and (c) applying one or more therapeutic treatments for a specific lysosomal storage disease to the subject if the level of CD63 in the sample is greater than the control value. In embodiments, the therapeutic treatment (e.g. the agent to be administered) comprises (e.g. consists of) a therapy of a type which is generally known to treat the disease in question, e.g. substrate reduction therapy, chaperone therapy, enzyme replacement therapy, and/or gene therapy.

CD63 levels can also be used to monitor the progression of a specific lysosomal storage disease, for example either to monitor a natural time course of the disease (e.g., to assess the right time to start a therapeutic intervention) or to monitor the impact of a therapeutic intervention on the disease (e.g., to assess the efficacy of a treatment, as a guide for dosage adjustment, etc.).

Thus, in one aspect is provided a method for monitoring the progress of a specific lysosomal storage disease in a subject diagnosed as having said disease, the method comprising comparing the level of CD63 in a first sample from the subject with the level of CD63 in a second sample from the subject, wherein the second sample was taken from the subject after the first sample was taken from the subject. Typically, the method will determine: that the disease is becoming more severe if the level of CD63 in the second sample is greater than the level of CD63 in the first sample; or that the disease in the subject is not progressing if the level of CD63 in the second sample is substantially the same as the level of CD63 in the first sample; or that the disease is becoming less severe if the level of CD63 in the second sample is lower than the level of CD63 in the first sample. A related aspect provides the use of CD63 as a biomarker for monitoring the progress of a specific lysosomal storage disease in a subject diagnosed as having said disease. Viewed in another way, these aspects provide a method for generating quantitative data for a subject diagnosed as having a specific lysosomal storage disease, the method comprising the steps of: (a) determining a level of CD63 in a first sample from the subject; (b) determining a level of CD63 in a subsequent sample from the subject; and (c) comparing the level of CD63 determined in step (a) with the level determined in step (b).

Another aspect provides a method for monitoring the progress of a treatment for a specific lysosomal storage disease in a subject diagnosed as having said disease, the method comprising: (a) measuring the level of CD63 in a first sample from the subject; (b) administering a therapeutic treatment for the specific lysosomal storage disease (e.g. a treatment as defined herein) to the subject; and (c) measuring the level of CD63 in a second sample from the subject, wherein the second sample was taken from the subject after the therapeutic treatment was administered. Typically, the method further comprises a step (d) of determining that the treatment is successful if the level of CD63 in the second sample is lower than the level of CD63 in the first sample. In embodiments, the dosage of the therapeutic treatment is to be increased if the level of CD63 determined in step (c) is substantially the same as, or greater than, the level of CD63 determined in step (a). A related aspect provides the use of CD63 as a biomarker for monitoring the progress of a treatment for a specific lysosomal storage disease in a subject diagnosed as having said disease. Viewed another way, these aspects provide a method for generating quantitative data for a subject having a specific lysosomal storage disease, the method comprising the steps of: (a) determining a level of CD63 in a first sample from the subject; and (b) determining a level of CD63 in a second sample from the subject after administration of a therapeutic treatment for said disease to the subject. In embodiments, the dosage of the therapeutic treatment is to be increased if the level of CD63 determined in step (b) is substantially the same as, or greater than, the level of CD63 determined in step (a).

Another aspect provides a method of treating a lysosomal storage disease in a patient in need thereof, wherein the patient has a higher than normal blood plasma CD63 level, e.g. a level of at least about 0.25 standard deviations (such as at least about 0.5, 0.75, 1, or 1.5 standard deviations) above the mean value for a healthy population of subjects. In embodiments, the method of treating comprises administering to the patient an effective amount of a therapeutic treatment capable of treating said lysosomal storage disease. In embodiments, the therapeutic treatment is a substrate reduction therapy and/or an enzyme replacement therapy (e.g. as described herein) capable of treating said lysosomal storage disease.

A normal blood plasma level can be determined, e.g., by a method as described herein (such as the Olink® assay described in detail in the Examples). In embodiments, the healthy population of subjects is a population having a similar genetic background, e.g., a population of the same heritage and/or geographical location as the patient. In embodiments, the lysosomal storage disease is Fabry disease, Gaucher disease (e.g. Gaucher disease type 1, type 2, or type 3), or MPS (e.g. MPS type I, II, or III).

A further aspect provides a method of treating a lysosomal storage disease in a subject diagnosed as being at risk of suffering from said lysosomal storage disease, wherein the patient has a blood plasma CD63 level which is higher than a control level in a sample previously taken from the subject, e.g. at least about 10% higher than (such as at least about 20%, 30%, 40%, 50%, 60%, 80%, or 100% higher than) the blood plasma control level. In embodiments, the method of treating comprises administering to the subject an effective amount of a substrate reduction therapy or an enzyme replacement therapy (e.g. as described herein) capable of treating said lysosomal storage disease.

In embodiments, the lysosomal storage disease is Fabry disease (e.g. wherein the subject is male), Gaucher disease (e.g. Gaucher disease type 1, type 2, or type 3), or MPS (e.g. MPS type I, II, or III). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level. In embodiments, the control value is derived from measuring the CD63 level in a single sample drawn from the subject. In other embodiments, the control value is an average is derived from measuring the CD63 level in multiple samples drawn from the subject (e.g. 2, 3, 4, 6, 8, or more samples).

Another aspect provides a method of treating or preventing the development or progression of a lysosomal storage disease in a subject assessed as being at risk of suffering from said disease, the method comprising the steps of: (a) taking a first biological sample (e.g. blood or a blood fraction such as plasma) from the subject and analyzing said sample for CD63 concentration; (b) if the CD63 concentration is above a control value, starting the subject on a course of therapeutic treatment (e.g. a therapeutically effective amount of substrate reduction therapy or an enzyme replacement therapy (e.g. as described herein) capable of treating said lysosomal storage disease), and optionally: (c) after treating the subject, taking a second biological sample (e.g. being the same type as the first biological sample) from the subject and analyzing said sample for CD63 concentration to determine a change in CD63 level; and (d) adjusting the therapeutic treatment based on the change in CD63 level observed.

In embodiments, the control value is about 10% higher (e.g. about 20%, 30%, 40%, 50%, 60%, 80%, or 100% higher than) than a control level in a sample previously taken from the same subject. In embodiments, the control level is derived from measuring the CD63 level in a single sample drawn from the subject. In other embodiments, the control value is an average is derived from measuring the CD63 level in multiple samples drawn from the subject (e.g. 2, 3, 4, 6, 8, or more samples). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level.

A further aspect provides a method of reducing CD63 levels in a patient suffering from a lysosomal storage disease (e.g. in the blood of the patient), the method comprising the step of administering an effective amount of substrate reduction therapy or an enzyme replacement therapy (e.g. as described herein). In embodiments, the lysosomal storage disease is Fabry disease, Gaucher disease (e.g. Gaucher disease type 1, type 2, or type 3), or MPS (e.g. MPS type I, II, or III). In embodiments, the reduction in CD63 is a reduction in blood plasma CD63 levels.

It will be appreciated that the various embodiments of the methods set out earlier may be applied to the above methods (e.g., as regards the selection of sample types for the test sample and control samples). In particular embodiments, the specific lysosomal storage disease is selected from the conditions listed above.

These methods are further illustrated below for Fabry disease, Gaucher disease and MPS, which are also the subject of the present Examples.

### Fabry Disease

Fabry disease (FD) is a lysosomal storage disorder characterized by deficient activity of α-Gal, encoded by the GLA gene. Enzyme deficiency results in the progressive intracellular accumulation of glycosphingolipids, mostly GL3, in a variety of cell types and tissues including kidney, heart, liver, spleen, and skin, as well as in the peripheral and central nervous systems. Symptoms presented by FD patients are highly variable and depend on the severity and stage of the condition. Symptoms usually begin during childhood or adolescence and may include: acroparesthesia (severe pain in the extremities, e.g., burning sensations in the arms and legs that get worse with exercise and hot weather), lenticular and corneal opacities, angiokeratoma, edema, abdominal pain, impaired blood circulation and increased risk of heart attack or stroke, enlarged heart, progressive kidney impairment (e.g., leading to renal failure), and decreased sweating, fever, and gastrointestinal difficulties. Renal, cardiac, and cerebrovascular symptoms tend to characterize later stages of the disease and are a significant cause of morbidity. Fabry disease is an X-linked lipid storage disease, with boys having a 50% chance of inheriting the disorder from their mother and daughters having a 50% chance of being a carrier. A milder form of the disease is common in females, although affected females may occasionally have severe symptoms similar to males with the disorder. Diagnosis has historically been made on a presumptive basis from considering clinical symptoms and family history, followed by assaying α-Gal activity in leukocytes or plasma, and/or detection of GL3 in tissue biopsies; the diagnosis could then be confirmed with molecular genetic analysis (Breunig et al., Kidney International (2003) 63(84):S181-S185). More recently, lyso-GL3 has been used as a diagnostic biomarker (Maruyama et al., Genet. Med. (2019) 21(1):44-52; see also Levstek et al., Genes (Basel) (2020) 11(9):1091-1109 for other diagnostics). Treatments for FD include ERT using recombinant α-Gal (e.g. agalsidase beta and agalsidase alfa), small molecule chaperone therapy (e.g. migalastat), and substrate reduction therapy (e.g. venglustat). Other therapeutic interventions are also under investigation (see, e.g., Oder et al., Cardiovasc Diagn Ther. (2021) 11(2):683-695; and also Lenders et al., Drugs (2021) 81(6):635-645).

As shown in the accompanying Examples, CD63 levels are increased in samples from Fabry disease patients as compared to matched healthy control subjects. In particular, it has now been observed that CD63 is upregulated in the plasma of Fabry patients; CD63 levels decrease with treatment in a linear manner (both with SRT and ERT); and the response of CD63 to treatment (SRT with venglustat) is comparable to the canonical Fabry biomarkers, GL3, and lyso-GL3 in its variability. Identifying CD63 as a biomarker for FD therefore represents an important advance in the diagnosis and monitoring of this disease.

Thus, in one aspect is provided a method of diagnosing Fabry disease in a subject suspected as being at risk of suffering from Fabry disease, the method comprising measuring the level of CD63 in a sample from the subject. A related aspect provides the use of CD63 as a biomarker in the diagnosis of Fabry disease in a subject suspected as being at risk of suffering from Fabry disease. Viewed in another way, these aspects provide a method for generating quantitative data for a subject, the method comprising determining the level of CD63 in a sample from the subject, wherein the subject is suspected of suffering from (or is suffering from) Fabry disease.

In embodiments, the subject is considered as being at risk of suffering from Fabry disease through the identification of specific risk factors such as family history of the disease, genetic testing, the analysis of other characteristic biomarkers, clinical symptoms, and the like, as well as those described herein. In embodiments, the specific risk factors are selected from one or more of clinical symptoms, family history, genetic testing, enzyme activity, and glycosphingolipid levels. The clinical symptoms may include one or more of fatigue, pain (e.g. acroparesthesia or abdominal pain), lenticular or corneal opacity, vortex keratopathy, angiokeratoma, shortness of breath, palpitations, edema, renal disease (e.g. proteinuria, microhematuria, or lipiduria, or end-stage renal disease), myocardial dysfunction (e.g. concentric or asymmetrical left ventricular hypertrophy, or heart failure), conduction abnormalities with reduced PR-interval, cardiac arrhythmias, vertigo, headache, diplopia, dysarthria, hemiataxia, transient ischemic attacks, premature stroke, and dementia. Family history may include history of Fabry disease among ancestors and/or close relatives (e.g., aunts, uncles, cousins, etc.; see, e.g., Laney et al., J Genet Couns. (2008) 17(1):79-83). Genetic testing may involve sequencing some or all of the GLA gene and comparing that with known disease-causing variants (see, e.g., “Fabry Disease: Perspectives from 5 Years of FOS”, Oxford PharmaGenesis (2006), Ed. Mehta, Beck and Sunder-Plassmann; Chapter 33). The mutational status of the patient may allow a diagnosis of Fabry disease to be made with a high degree of confidence, e.g., in the case of some mutations which result in premature termination of enzyme translation. However, in other cases, it may not be possible to correlate a variant GLA with Fabry disease absolutely. Enzyme activity may be assessed by testing for the activity of α-Gal, e.g. in a whole blood or blood spot sample (see, e.g., https://www.discoverfabry.com/hcp/diagnosing-fabry; and also Nakao et al., Kidney Int. (2003) 64(3):801-807). Glycosphingolipid levels may be assessed by measuring the concentration of GL3 and/or lyso-GL3 in a sample, e.g. a whole blood or plasma sample (see, e.g., Maruyama et al., 2019, supra).

In embodiments, the control value is a baseline CD63 level which has previously been measured in the same subject (i.e. in an earlier sample from the subject), for example measured at least 1 month prior to obtaining the test sample from the subject, e.g. at least 2, 3, 6, 9, 12, 18, or 24 months prior to obtaining the test sample from the subject. The control value may represent the CD63 level measured in a single earlier sample from the subject or it may be an average of values from multiple earlier samples. In an alternative embodiment, the control value is the CD63 level in a sample taken from one or more healthy subjects (e.g. an average value for a cohort of healthy subjects). In embodiments, the healthy subjects are matched to the subject being assessed, e.g. by age and/or sex. The healthy subjects are generally matched to the subject being assessed by sex. Typically, the control value represents the level of CD63 in the same type of sample which is being assessed in the present methods, e.g., a sample which has been obtained, processed, and/or stored in the same way as the test sample. For example, where the present method utilizes a plasma sample, the control value typically represents the level of CD63 in a control plasma sample, e.g., an earlier plasma sample taken from the same subject or a plasma sample obtained from one or more healthy subjects.

In embodiments where the control value for CD63 concentration is a point value (or an average, e.g. mean, value), the CD63 level in the test sample is considered to be outside the normal range if it is at least about 5% greater than the control value, e.g. at least about 7.5%, 10%, 12.5%, 15%, 20%, 25%, 30%, 40%, 50%, 75%, or 100% greater than the control value. In some embodiments, the CD63 level in the test sample is considered to be outside the normal range if it is at least about 2 times greater than the control value, e.g. at least about 3, 4, 5, 6, 8, or 10 times the control value. In embodiments, the CD63 level in the test sample is from about 3 to about 8 times the control value, e.g. from about 4 to about 7 times the control value, or from about 5 to about 6 times the control value. In embodiments, the CD63 level in the test sample is about 5 times the control value, or about 6 times the control value. In embodiments where the control value for CD63 concentration is a range of values, e.g. described by some variability around a mean value, the CD63 level in the test sample is considered to be outside the normal range if it is more than about 1 standard error greater than the mean value, e.g. more than about 1.0, 1.1, 1.2, 1.3, 1.4, 1.5, 1.6, 1.7, 1.8, 1.9, 2.0, 2.5, or 3.0 standard errors greater than the mean value.

In embodiments, CD63 is used as the only biomarker in the methods of the disclosure.

Alternatively, measurement of the CD63 level in the sample from the subject is used alongside one or more other characteristic measures of the disease, e.g., as part of a panel of biomarkers used for diagnostic purposes. This aspect provides a method of improving the diagnosis of Fabry disease in a subject, characterized in that CD63 is used as a biomarker. A related aspect provides the use of CD63 as a biomarker to improve a method of diagnosing Fabry disease in a subject. In embodiments, CD63 is used as a biomarker alongside GL3, lyso-GL3, and/or α-Gal activity, optionally with one or more further biomarkers. In embodiments, CD63 is used as a biomarker alongside lyso-GL3, optionally with one or more further biomarkers. In embodiments, the other biomarkers used (i.e., besides CD63) do not include any of the following: α-iduronidase, α-glucosidase, saposin C, LAMP-1, LAMP-2, β-glucosidase, α-galactosidase A, iduronate-2-sulphatase, N-acetylgalactosamine 4-sulphatase, galactose 6-sulphatase, acid sphingomyelinase, galactocerebrosidase, arylsulphatase A, saposin B, heparan-N-sulphatase, α-N-acetylglucosaminidase, acetylCoA:glucosamine N-acetyltransferase, N-acetylglucosamine 6-sulphatase, β-galactosidase, β-glucuronidase, aspartylglucosaminidase, acid lipase, β-hexosamindase A, β-hexosamindase B, GM2-activator, acid ceramidase, α-L-fucosidase, α-D-mannosidase, β-D-mannosidase, neuraminidase, phosphotransferase, phosphotransferase g-subunit, palmitoyl protein thioesterase, tripeptidyl peptidase I, cathespsin K, α-galactosidase B, sialic acid transporter, CD45 leukocyte common biomolecule, or LIMP II.

In embodiments, the use of CD63 as a biomarker reduces the proportion of false positives and/or increases the proportion of true positives as compared to a corresponding diagnosis in which the CD63 level is not measured.

Another aspect provides a method of treating a subject who has been diagnosed as having Fabry disease by a method as defined herein, the treatment comprising administering to the subject one or more therapeutic treatments for Fabry disease. A related aspect provides a therapeutic agent for the treatment of Fabry disease in a subject, wherein the subject has been diagnosed as having Fabry disease by a method as defined herein. Also provided is a method for generating quantitative data for a subject, wherein the method comprises (e.g. consists of): (a) determining a level of CD63 in a sample from the subject; (b) determining whether the level of CD63 is greater than a control value (e.g., a control value measured as the CD63 level in a sample taken from one or more healthy subjects); and (c) applying one or more therapeutic treatments for Fabry disease to the subject if the level of CD63 in the sample is greater than the control value.

In embodiments, the therapeutic treatment or agent comprises (e.g. consists of) substrate reduction therapy (e.g. venglustat), chaperone therapy (e.g. migalastat), enzyme replacement therapy (e.g. agalsidase alfa or agalsidase beta), and/or gene therapy (e.g. using the GLA gene or active fragments thereof). In embodiments, the therapeutic treatment comprises (e.g. consists of) administering venglustat or migalastat (e.g. venglustat) to the subject. In other embodiments, the treatment comprises administering recombinant α-galactosidase (e.g. agalsidase beta) to the subject.

A further aspect provides a method for monitoring the progress of Fabry disease in a subject diagnosed as having Fabry disease, the method comprising: (a) measuring the level of CD63 in a first sample from the subject; (b) measuring the level of CD63 in a second sample from the subject, wherein the second sample was taken from the subject after the first sample was taken from the subject; and (c) comparing the level of CD63 in the first sample with the level of CD63 in the second sample. Typically, the method further comprises a step (d) determining that the disease is becoming more severe if the level of CD63 in the second sample is greater than the level of CD63 in the first sample, determining that the disease in the subject is not progressing if the level of CD63 in the second sample is substantially the same as the level of CD63 in the first sample, and determining that the disease is becoming less severe if the level of CD63 in the second sample is lower than the level of CD63 in the first sample. A related aspect provides the use of CD63 as a biomarker for monitoring the progress of Fabry disease in a subject diagnosed as having Fabry disease. Viewed in another way, these aspects provide a method for generating quantitative data for a subject diagnosed as having Fabry disease, the method comprising the steps of: (a) determining a level of CD63 in a first sample from the subject; (b) determining a level of CD63 in a subsequent sample from the subject; and (c) comparing the level of CD63 determined in step (a) with the level determined in step (b). The method may comprise a further step (d) in which the progress of the disease is assessed based on the comparison carried out in step (c).

The sample may be of a type mentioned herein. In embodiments, the sample contains lysosomes and/or exosomes. In embodiments, the sample is selected from blood, a blood fraction, urine, cerebrospinal fluid, sputum, lymph, dermal tissue, renal tissue, cardiac tissue, spleen tissue, bone marrow, and the like. Most conveniently, the sample from the subject comprises (e.g. consists of) blood (e.g. whole blood), a blood fraction, and/or urine. In embodiments, the sample from the subject comprises (e.g. consists of) a blood fraction selected from plasma and serum. In embodiments, the sample is a blood sample, e.g. a plasma sample. In embodiments, the sample comprises (e.g., consists of) CSF.

In embodiments, the first and second (or subsequent) samples are taken from the subject at an interval of at least about 1 day, e.g. at least about 2, 3, or 4 days, or at least about 1, 2, or 4 weeks. In other embodiments, the first and second (or subsequent) samples are taken from the subject at an interval of at least about 8 weeks, e.g. at least about 9, 10, 12, 15, or 20 weeks.

In embodiments, the methods comprise the ongoing measurement of one or more further samples taken from the subject, e.g., at the intervals specified above.

Another aspect provides a method for monitoring the progress of a treatment for Fabry disease in a subject diagnosed as having Fabry disease, the method comprising: (a) measuring the level of CD63 in a first sample from the subject; (b) administering a therapeutic treatment for Fabry disease to the subject; and (c) measuring the level of CD63 in a second sample from the subject, wherein the second sample was taken from the subject after the therapeutic treatment was administered. Typically, the method further comprises a step (d) of determining that the treatment is successful if the level of CD63 in the second sample is lower than the level of CD63 in the first sample. In embodiments, the dosage (e.g., the amount and/or the frequency of administration) of the therapeutic treatment is to be increased if the level of CD63 determined in step (c) is substantially the same as, or greater than, the level of CD63 determined in step (a). A related aspect provides the use of CD63 as a biomarker for monitoring the progress of a treatment for Fabry disease in a subject diagnosed as having Fabry disease. Viewed another way, these aspects provide a method for generating quantitative data for a subject having Fabry disease, the method comprising the steps of: (a) determining a level of CD63 in a first sample from the subject; and (b) determining a level of CD63 in a second sample from the subject after administration of a therapeutic treatment for Fabry disease to the subject. In embodiments, the dosage of the therapeutic treatment is to be increased if the level of CD63 determined in step (b) is substantially the same as, or greater than, the level of CD63 determined in step (a).

In embodiments, the treatment for Fabry disease is as described herein. Thus, the treatment may comprise (e.g. consist of) substrate reduction therapy, chaperone therapy, enzyme replacement therapy or gene therapy. In embodiments, the treatment comprises administering venglustat or migalastat (e.g. venglustat) to the subject. In other embodiments, the treatment comprises administering recombinant α-galactosidase (e.g. agalsidase beta) to the subject.

The second sample is typically taken after a period of time has elapsed from the administration of the therapeutic treatment. In embodiments, the second sample is taken from the subject at least about 1 week after administration (e.g., after initiation) of the therapeutic treatment, e.g. at least about 2, 3, 4, 6, 8, 10, 12, 15, or 20 weeks after administration of the therapeutic treatment. In one embodiment, the second sample is taken from the subject at least about 8 weeks after initiation of the therapeutic treatment.

In embodiments, the methods comprise the ongoing measurement of one or more further samples taken from the subject, e.g., at intervals of about every about 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method of treating Fabry disease in a patient in need thereof, wherein the patient has a higher than normal blood plasma CD63 level (e.g. a level of at least about 0.25 standard deviations, such as at least about 0.5, 0.75, 1, or 1.5 standard deviations) above the mean value for a healthy population of subjects, the method comprising administering to the patient an effective amount of a therapeutic treatment for Fabry disease. In embodiments, the healthy population of subjects is a population having a similar genetic background, e.g., a population of the same heritage and/or geographical location as the patient.

A further aspect provides a method of treating Fabry disease in a subject diagnosed as being at risk of developing said disease, wherein the patient has a blood plasma CD63 level which is higher than a control level in a sample previously taken from the subject (e.g. at least about 10% higher than, such as at least about 20%, 30%, 40%, 50%, 60%, 80%, or 100% higher than, the blood plasma control level), the method comprising administering to the subject an effective amount of a therapeutic treatment for Fabry disease (e.g. as described herein). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level. In embodiments, the control value is derived from measuring the CD63 level in a single sample drawn from the subject. In other embodiments, the control value is an average is derived from measuring the CD63 level in multiple samples drawn from the subject (e.g. 2, 3, 4, 6, 8, or more samples).

A further aspect provides a method of reducing CD63 levels in a patient suffering from Fabry disease (e.g. in the blood of the patient), the method comprising the step of administering an effective amount of a therapeutic treatment (e.g. a substrate reduction therapy or an enzyme replacement therapy as described herein) to the patient. In embodiments, the reduction in CD63 is a reduction in blood plasma CD63 levels.

Another aspect provides a method of treating or preventing the development or progression of Fabry disease in a subject assessed as being at risk of suffering from Fabry disease, the method comprising the steps of: (a) taking a first biological sample (e.g. blood or a blood fraction such as plasma) from the subject and analyzing said sample for CD63 concentration; (b) if the CD63 concentration is above a control value, starting the subject on a course of therapeutic treatment (e.g. a therapeutically effective amount of substrate reduction therapy or an enzyme replacement therapy, such as those described herein), and optionally: (c) after treating the subject, taking a second biological sample (e.g. being the same type as the first biological sample) from the subject and analyzing said sample for CD63 concentration to determine a change in CD63 level; and (d) adjusting the therapeutic treatment based on the change in CD63 level observed.

In embodiments, the control value is about 10% higher (e.g. about 20%, 30%, 40%, 50%, 60%, 80%, or 100% higher than) than a control level in a sample previously taken from the same subject. In embodiments, the control level is derived from measuring the CD63 level in a single sample drawn from the subject. In other embodiments, the control value is an average is derived from measuring the CD63 level in multiple samples drawn from the subject (e.g. 2, 3, 4, 6, 8, or more samples). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level.

In embodiments, the treatment for Fabry disease is as described herein. Thus, the treatment may comprise (e.g. consist of) substrate reduction therapy, chaperone therapy, enzyme replacement therapy, or gene therapy. In embodiments, the treatment comprises administering venglustat or migalastat (e.g. venglustat) to the subject. In other embodiments, the treatment comprises administering recombinant α-galactosidase (e.g. agalsidase beta) to the subject.

The second sample is typically taken after a period of time has elapsed from the administration of the therapeutic treatment. In embodiments, the second sample is taken from the subject at least about 1 week after administration (e.g., after initiation) of the therapeutic treatment, e.g. at least about 2, 3, 4, 6, 8, 10, 12, 15, or 20 weeks after administration of the therapeutic treatment. In one embodiment, the second sample is taken from the subject at least about 8 weeks after initiation of the therapeutic treatment.

In embodiments, the methods comprise the ongoing measurement of one or more further samples taken from the subject, e.g., at intervals of about every about 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method for adjusting the dosage of a therapeutic treatment for Fabry disease in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample. Typically, the dosage adjustment in step (c) comprises maintaining the dosage of the therapeutic treatment if the level of CD63 in the second sample is lower than the level of CD63 in the first sample and increasing the dosage (e.g., the amount and/or the frequency of administration) of the therapeutic treatment if the level of CD63 in the second sample is substantially the same as, or greater than, the level of CD63 in the first sample. In embodiments, the dosage (e.g., the amount and/or the frequency of administration) of the therapeutic treatment is decreased if the level of CD63 in the second sample is lower than the level of CD63 in the first sample. A related aspect provides the use of CD63 as a biomarker for adjusting the dosage of a therapeutic treatment for Fabry disease in a subject receiving said therapeutic treatment.

In embodiments, the subject is a male subject. In other embodiments, the subject is a female subject.

### Gaucher Disease

Gaucher disease (GD) is an inherited metabolic disorder caused by mutations in the GBA gene which result in deficiency of GCase (also called acid β-glucosidase) and an associated accumulation of GL1 and lyso-GL1 in the lysosomes of macrophages; this affects cells of the reticuloendothelial system, including liver, spleen, and bone marrow, which can lead to enlarged spleen and liver, liver malfunction, skeletal disorders, bone lesions, or severe neurological complications. The forms of GD are grouped into three main types—Type 1 GD is a non-neuropathic form which is the most common type and chiefly affects adults, having an average age at diagnosis of around 28 years; Type 2 GD is an acute neuronopathic form which typically affects infants in the first 3-6 months of life; and Type 3 is a chronic neuronopathic form which has a later and more gradual onset than Type 2. Gaucher disease type 3 (GD3) is characterized by progressive encephalopathy and systemic manifestations, similar to type 1. It is caused by a mutation in the GBA gene (1q21) and it features prominent central nervous system (CNS) involvement which poses significant challenges for diagnosis and monitoring.

Lyso-GL1 levels, β-glucosidase activity, and genetic GBA sequencing are currently the most common diagnostic and prognostic indicators for GD. In the case of lyso-GL1, some studies suggest that its pathological involvement is correlated with disease burden and clinical severity. Chitotriosidase and CCL18 have also been identified as biomarkers for Gaucher disease but are not commonly used in the clinic (see, e.g., Rolfs et al., PLoS ONE (2013) 8(11):e79732).

As shown in the accompanying Examples, CD63 levels are elevated in Gaucher patients and show a much lower variability in treated patients than canonical biomarkers for GD such as lyso-GL1 or chitotriosidase. CD63 therefore represents a valuable biomarker for use in diagnosing and monitoring GD.

Thus, in one aspect is provided a method of diagnosing Gaucher disease in a subject suspected as being at risk of suffering from Gaucher disease, the method comprising measuring the level of CD63 in a sample from the subject. A related aspect provides the use of CD63 as a biomarker in the diagnosis of Gaucher disease in a subject suspected as being at risk of suffering from Gaucher disease. Viewed in another way, these aspects provide a method for generating quantitative data for a subject, the method comprising determining the level of CD63 in a sample from the subject, wherein the subject is suspected of suffering from (or is suffering from) Gaucher disease. In embodiments, the Gaucher disease is type 1 GD, type 2 GD, or type 3 GD. In embodiments, the Gaucher disease is type 3 GD.

In embodiments, the subject is considered as being at risk of suffering from Gaucher disease (e.g. type 1 GD) through the identification of specific risk factors such as family history of the disease, genetic testing, the analysis of other characteristic biomarkers, clinical symptoms, and the like, as well as those described herein. In embodiments, the specific risk factors are selected from one or more of clinical symptoms, family history, genetic testing, enzyme activity, and glycosphingolipid levels. The clinical symptoms may include one or more of hepatomegaly and splenomegaly, pain (especially severe pain of the joints, e.g. hips and/or knees), osteoporosis, skin pigmentation, pancytopenia (e.g., leading to anemia, neutropenia, leukopenia, and/or thrombocytopenia, with an increased risk of infection and bleeding), and neurological symptoms (e.g., impaired olfaction and cognition, especially in type 1 GD; convulsions, hypertonia, intellectual disability, and apnea, especially in type 2 GD; and myoclonus, convulsions, dementia, and ocular muscle apraxia, especially in type 3 GD), as well as parkinsonism. Family history may include history of Gaucher disease among ancestors and/or close relatives (e.g., aunts, uncles, cousins, etc.). Genetic testing may involve sequencing some or all of the GBA gene and comparing that with known disease-causing variants (see, e.g., Riboldi et al., Cells (2019) 8:364-380). The mutational status of the patient may allow a diagnosis of Gaucher disease to be made with a high degree of confidence, e.g., in the case of mutations which are known to be associated with severe disease. However, in other cases, it may not be possible to correlate a variant GBA with Gaucher disease absolutely. Enzyme activity may be assessed by testing for the activity of GCase, e.g. in a whole blood or blood spot sample (see, e.g., Miyamoto et al., Intern Med. (2021) 60:699-707). Glycosphingolipid levels may be assessed by measuring the concentration of GL1 and/or lyso-GL1 in a sample, e.g. a whole blood or plasma sample (see, e.g., Rolfs et al., 2013, supra).

In embodiments, the control value is a baseline CD63 level which has previously been measured in the same subject (i.e. in an earlier sample from the subject), for example measured at least 1 month prior to obtaining the test sample from the subject, e.g. at least 2, 3, 6, 9, 12, 18, or 24 months prior to obtaining the test sample from the subject. The control value may represent the CD63 level measured in a single earlier sample from the subject or it may be an average of values from multiple earlier samples. In an alternative embodiment, the control value is the CD63 level in a sample taken from one or more healthy subjects (e.g., an average value for a cohort of healthy subjects). In embodiments, the healthy subjects are matched to the subject being assessed, e.g. by age and/or sex. Typically, the control value represents the level of CD63 in the same type of sample which is being assessed in the present methods, e.g., a sample which has been obtained, processed and/or stored in the same way as the test sample. For example, where the present method utilizes a plasma sample, the control value typically represents the level of CD63 in a control plasma sample, e.g., an earlier plasma sample taken from the same subject or a plasma sample obtained from one or more healthy subjects.

In embodiments where the control value for CD63 concentration is a point value (or an average, e.g. mean, value), the CD63 level in the test sample is considered to be outside the normal range if it is at least about 5% greater than the control value, e.g. at least about 7.5%, 10%, 12.5%, 15%, 20%, 25%, 30%, 40%, 50%, 75%, or 100% greater than the control value. In some embodiments, the CD63 level in the test sample is considered to be outside the normal range if it is at least about 2 times greater than the control value, e.g. at least about 3, 4, 5, 6, 8, or 10 times the control value. In embodiments, the CD63 level in the test sample is from about 3 to about 8 times the control value, e.g. from about 4 to about 6 times the control value, or from about 4 to about 5 times the control value. In embodiments, the CD63 level in the test sample is about 4 times the control value, or about 5 times the control value. In embodiments where the control value for CD63 concentration is a range of values, e.g. described by some variability around a mean value, the CD63 level in the test sample is considered to be outside the normal range if it is more than about 1 standard error greater than the mean value, e.g. more than about 1.0, 1.1, 1.2, 1.3, 1.4, 1.5, 1.6, 1.7, 1.8, 1.9, 2.0, 2.5, or 3.0 standard errors greater than the mean value.

In embodiments, CD63 is used as the only biomarker in the methods of the disclosure.

Alternatively, measurement of the CD63 level in the sample from the subject is used alongside one or more other characteristic measures of the disease, e.g., as part of a panel of biomarkers used for diagnostic purposes. This aspect provides a method of improving the diagnosis of Gaucher disease in a subject, characterized in that CD63 is used as a biomarker. A related aspect provides the use of CD63 as a biomarker to improve a method of diagnosing Gaucher disease in a subject. In embodiments, the Gaucher disease is type 1 GD, type 2 GD, or type 3 GD. In embodiments, CD63 is used as a biomarker alongside GL1, lyso-GL1, CCL18, and/or chitotriosidase, optionally with one or more further biomarkers. In embodiments, the other biomarkers used (i.e., besides CD63) do not include any of the following: α-iduronidase, α-glucosidase, saposin C, LAMP-1, LAMP-2, β-glucosidase, α-galactosidase A, iduronate-2-sulphatase, N-acetylgalactosamine 4-sulphatase, galactose 6-sulphatase, acid sphingomyelinase, galactocerebrosidase, arylsulphatase A, saposin B, heparan-N-sulphatase, α-N-acetylglucosaminidase, acetylCOA:glucosamine N-acetyltransferase, N-acetylglucosamine 6-sulphatase, β-galactosidase, β-glucuronidase, aspartylglucosaminidase, acid lipase, β-hexosamindase A, β-hexosamindase B, GM2-activator, acid ceramidase, α-L-fucosidase, α-D-mannosidase, β-D-mannosidase, neuraminidase, phosphotransferase, phosphotransferase g-subunit, palmitoyl protein thioesterase, tripeptidyl peptidase I, cathespsin K, α-galactosidase B, sialic acid transporter, CD45 leukocyte common biomolecule, or LIMP II.

In embodiments, the use of CD63 as a biomarker reduces the proportion of false positives and/or increases the proportion of true positives as compared to a corresponding diagnosis in which the CD63 level is not measured.

Another aspect provides a method of treating a subject who has been diagnosed as having Gaucher disease by a method as defined herein, the treatment comprising administering to the subject one or more therapeutic treatments for Gaucher disease. A related aspect provides a therapeutic agent for the treatment of Gaucher disease in a subject, wherein the subject has been diagnosed as having Gaucher disease by a method as defined herein. Also provided is a method for generating quantitative data for a subject, wherein the method comprises (e.g. consists of): (a) determining a level of CD63 in a sample from the subject; (b) determining whether the level of CD63 is greater than a control value (e.g., a control value measured as the CD63 level in a sample taken from one or more healthy subjects); and (c) applying one or more therapeutic treatments for Gaucher disease to the subject if the level of CD63 in the sample is greater than the control value. In embodiments, the Gaucher disease is type 1 GD, type 2 GD, or type 3 GD.

In embodiments, the therapeutic treatment or agent comprises (e.g. consists of) substrate reduction therapy (e.g. eliglustat, venglustat, or miglustat), chaperone therapy, enzyme replacement therapy (e.g. imiglucerase, velaglucerase, or taliglucerase), and/or gene therapy (e.g. using the GBA gene). In embodiments, the therapeutic treatment comprises (e.g. consists of) administering eliglustat to the subject. In embodiments, the therapeutic treatment comprises (e.g. consists of) administering venglustat to the subject. In other embodiments, the treatment comprises administering recombinant glucocerebrosidase (e.g. imiglucerase) to the subject. In embodiments, the therapeutic treatment comprises (e.g. consists of) administering venglustat and recombinant glucocerebrosidase (e.g. imiglucerase) to the subject. In embodiments, the Gaucher disease is type 1 GD and the therapeutic treatment comprises (e.g. consists of) administering eliglustat to the subject. In embodiments, the Gaucher disease is type 2 or type 3 GD and the therapeutic treatment comprises (e.g. consists of) administering venglustat to the subject. In embodiments, the Gaucher disease is type 3 GD and the therapeutic treatment comprises (e.g. consists of) administering venglustat and recombinant glucocerebrosidase (e.g. imiglucerase) to the subject. In embodiments, the Gaucher disease is type 3 GD and the therapeutic treatment comprises (e.g. consists of) administering venglustat to the subject.

A further aspect provides a method for monitoring the progress of Gaucher disease in a subject diagnosed as having Gaucher disease, the method comprising: (a) measuring the level of CD63 in a first sample from the subject; (b) measuring the level of CD63 in a second sample from the subject, wherein the second sample was taken from the subject after the first sample was taken from the subject; and (c) comparing the level of CD63 in the first sample with the level of CD63 in the second sample. Typically, the method further comprises a step (d) determining that the disease is becoming more severe if the level of CD63 in the second sample is greater than the level of CD63 in the first sample, determining that the disease in the subject is not progressing if the level of CD63 in the second sample is substantially the same as the level of CD63 in the first sample, and determining that the disease is becoming less severe if the level of CD63 in the second sample is lower than the level of CD63 in the first sample. A related aspect provides the use of CD63 as a biomarker for monitoring the progress of Gaucher disease in a subject diagnosed as having Gaucher disease. Viewed in another way, these aspects provide a method for generating quantitative data for a subject diagnosed as having Gaucher disease, the method comprising the steps of: (a) determining a level of CD63 in a first sample from the subject; (b) determining a level of CD63 in a subsequent sample from the subject; and (c) comparing the level of CD63 determined in step (a) with the level determined in step (b). The method may comprise a further step (d) in which the progress of the disease is assessed based on the comparison carried out in step (c). In embodiments, the Gaucher disease is type 1 GD, type 2 GD, or type 3 GD.

The sample may be of a type mentioned herein. In embodiments, the sample contains lysosomes and/or exosomes. In embodiments, the sample is selected from blood, a blood fraction, urine, cerebrospinal fluid, sputum, lymph, dermal tissue, renal tissue, cardiac tissue, spleen tissue, bone marrow, and the like. Conveniently, the sample from the subject comprises (e.g. consists of) blood (e.g. whole blood), a blood fraction, and/or urine. In embodiments, the sample from the subject comprises (e.g. consists of) a blood fraction selected from plasma and serum. In embodiments, the sample is a blood sample, e.g. a plasma sample. In embodiments, the sample is a CSF sample. In embodiments, the sample does not comprise platelets.

In embodiments, the first and second (or subsequent) samples are taken from the subject at an interval of at least about 1 day, e.g. at least about 2, 3, or 4 days, or at least about 1, 2, or 4 weeks. In other embodiments, the first and second (or subsequent) samples are taken from the subject at an interval of at least about 8 weeks, e.g. at least about 9, 10, 12, 15, or 20 weeks.

In embodiments, the methods comprise the ongoing measurement of one or more further samples taken from the subject, e.g., at the intervals specified above.

Another aspect provides a method for monitoring the progress of a treatment for a Gaucher disease in a subject diagnosed as having Gaucher disease, the method comprising: (a) measuring the level of CD63 in a first sample from the subject; (b) administering a therapeutic treatment for Gaucher disease to the subject; and (c) measuring the level of CD63 in a second sample from the subject, wherein the second sample was taken from the subject after the therapeutic treatment was administered. Typically, the method further comprises a step (d) of determining that the treatment is successful if the level of CD63 in the second sample is lower than the level of CD63 in the first sample. In embodiments, the dosage (e.g., the amount and/or the frequency of administration) of the therapeutic treatment is to be increased if the level of CD63 determined in step (c) is substantially the same as, or greater than, the level of CD63 determined in step (a). A related aspect provides the use of CD63 as a biomarker for monitoring the progress of a treatment for Gaucher disease in a subject diagnosed as having Gaucher disease. Viewed another way, these aspects provide a method for generating quantitative data for a subject having Gaucher disease, the method comprising the steps of: (a) determining a level of CD63 in a first sample from the subject; and (b) determining a level of CD63 in a second sample from the subject after administration of a therapeutic treatment for Gaucher disease to the subject. In embodiments, the dosage of the therapeutic treatment is to be increased if the level of CD63 determined in step (b) is substantially the same as, or greater than, the level of CD63 determined in step (a). In embodiments, the Gaucher disease is type 1 GD, type 2 GD, or type 3 GD. In embodiments, the Gaucher disease is type 3 GD and the sample comprises (e.g., consists of) CSF.

In embodiments, the treatment for Gaucher disease is as described herein. Thus, the treatment may comprise (e.g. consist of) substrate reduction therapy, enzyme replacement therapy, and/or gene therapy. In embodiments, the treatment comprises administering eliglustat or miglustat (e.g. eliglustat) to the subject. In other embodiments, the treatment comprises administering recombinant glucocerebrosidase (e.g. imiglucerase) to the subject. In embodiments, the method monitors a change from a first treatment (such as ERT, e.g. with imiglucerase) to a second treatment (such as SRT, e.g. with eliglustat), for example to check that the patient improves or remains stable after the change.

The second sample is typically taken after a period of some time has elapsed from the administration of the therapeutic treatment. In embodiments, the second sample is taken from the subject at least about 1 week after administration (e.g., after initiation) of the therapeutic treatment, e.g. at least about 2, 3, 4, 6, 8, 10, 12, 15, or 20 weeks after administration of the therapeutic treatment. In one embodiment, the second sample is taken from the subject at least 8 weeks after initiation of the therapeutic treatment.

In embodiments, the methods comprise the ongoing measurement of one or more further samples taken from the subject, e.g., at intervals of about every 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method of treating Gaucher disease in a patient in need thereof, wherein the patient has a higher than normal blood plasma CD63 level (e.g. a level of at least about 0.25 standard deviations, such as at least about 0.5, 0.75, 1, or 1.5 standard deviations) above the mean value for a healthy population of subjects, the method comprising administering to the patient an effective amount of a therapeutic treatment for Gaucher disease. In embodiments, the healthy population of subjects is a population having a similar genetic background, e.g., a population of the same heritage and/or geographical location as the patient.

A further aspect provides a method of treating Gaucher disease in a subject diagnosed as being at risk of developing said disease, wherein the patient has a blood plasma CD63 level which is higher than a control level in a sample previously taken from the subject (e.g. at least about 10% higher than, such as at least about 20%, 30%, 40%, 50%, 60%, 80%, or 100% higher than, the blood plasma control level), the method comprising administering to the subject an effective amount of a therapeutic treatment for Gaucher disease (e.g. as described herein). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level. In embodiments, the control value is derived from measuring the CD63 level in a single sample drawn from the subject. In other embodiments, the control value is an average is derived from measuring the CD63 level in multiple samples drawn from the subject (e.g. 2, 3, 4, 6, 8, or more samples).

A further aspect provides a method of reducing CD63 levels in a patient suffering from Gaucher disease (e.g. in the blood of the patient), the method comprising the step of administering an effective amount of a therapeutic treatment (e.g. a substrate reduction therapy or an enzyme replacement therapy as described herein) to the patient. In embodiments, the reduction in CD63 is a reduction in blood plasma CD63 levels.

Another aspect provides a method of treating or preventing the development or progression of Gaucher disease in a subject assessed as being at risk of suffering from Gaucher disease, the method comprising the steps of: (a) taking a first biological sample (e.g. blood or a blood fraction such as plasma) from the subject and analyzing said sample for CD63 concentration; (b) if the CD63 concentration is above a control value, starting the subject on a course of therapeutic treatment (e.g. a therapeutically effective amount of substrate reduction therapy and/or an enzyme replacement therapy, such as those described herein), and optionally: (c) after treating the subject, taking a second biological sample (e.g. being the same type as the first biological sample) from the subject and analyzing said sample for CD63 concentration to determine a change in CD63 level; and (d) adjusting the therapeutic treatment based on the change in CD63 level observed.

In embodiments, the control value is about 10% higher (e.g. about 20%, 30%, 40%, 50%, 60%, 80%, or 100% higher than) than a control level in a sample previously taken from the same subject. In embodiments, the control level is derived from measuring the CD63 level in a single sample drawn from the subject. In other embodiments, the control value is an average is derived from measuring the CD63 level in multiple samples drawn from the subject (e.g. 2, 3, 4, 6, 8, or more samples). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level.

In embodiments, the treatment for Gaucher disease is as described herein. Thus, the treatment may comprise (e.g. consist of) substrate reduction therapy, chaperone therapy, enzyme replacement therapy, and/or gene therapy. In embodiments, the treatment comprises administering venglustat, eliglustat, or miglustat to the subject (e.g. venglustat or eliglustat). In other embodiments, the treatment comprises administering recombinant glucocerebrosidase (e.g. imiglucerase) to the subject. In embodiments, the therapeutic treatment comprises (e.g. consists of) administering venglustat and recombinant glucocerebrosidase (e.g. imiglucerase) to the subject. In embodiments, the Gaucher disease is type 1 GD and the therapeutic treatment comprises (e.g. consists of) administering eliglustat to the subject. In embodiments, the Gaucher disease is type 2 or type 3 GD and the therapeutic treatment comprises (e.g. consists of) administering venglustat to the subject. In embodiments, the Gaucher disease is type 3 GD and the therapeutic treatment comprises (e.g. consists of) administering venglustat and recombinant glucocerebrosidase (e.g. imiglucerase) to the subject. In embodiments, the Gaucher disease is type 3 GD and the therapeutic treatment comprises (e.g. consists of) administering venglustat to the subject.

The second sample is typically taken after a period of time has elapsed from the administration of the therapeutic treatment. In embodiments, the second sample is taken from the subject at least about 1 week after administration (e.g., after initiation) of the therapeutic treatment, e.g. at least about 2, 3, 4, 6, 8, 10, 12, 15, or 20 weeks after administration of the therapeutic treatment. In one embodiment, the second sample is taken from the subject at least about 8 weeks after initiation of the therapeutic treatment.

In embodiments, the methods comprise the ongoing measurement of one or more further samples taken from the subject, e.g., at intervals of about every about 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method for adjusting the dosage of a therapeutic treatment for Gaucher disease in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample. Typically, the dosage adjustment in step (c) comprises maintaining the dosage of the therapeutic treatment if the level of CD63 in the second sample is lower than the level of CD63 in the first sample and increasing the dosage (e.g., the amount and/or the frequency of administration) of the therapeutic treatment if the level of CD63 in the second sample is substantially the same as, or greater than, the level of CD63 in the first sample. In embodiments, the dosage (e.g., the amount and/or the frequency of administration) of the therapeutic treatment is decreased if the level of CD63 in the second sample is lower than the level of CD63 in the first sample. A related aspect provides the use of CD63 as a biomarker for adjusting the dosage of a therapeutic treatment for Gaucher disease in a subject receiving said therapeutic treatment.

### Mucopolysaccharidoses

The mucopolysaccharidoses are inherited disorders in which GAGs accumulate in connective and other tissues throughout the body as a result of deficiencies in lysosomal enzymes which degrade GAGs. Symptoms vary depending on the severity of the disease, but typically result from damage to the bone, connective tissues, and organs (e.g., dysplasia, joint stiffness, and hepatomegaly/splenomegaly) as well as from the resulting compression of nerves or nerve roots of the spinal cord (e.g., pain, impaired motor function, and other complications of the peripheral nervous system). There are currently seven clinical types of MPS identified, and multiple subtypes, classified according to the gene which is affected (see, e.g., Celik et al., Diagnostics (2021) 11(2):273-311). Current treatment options include enzyme replacement therapy (ERT) and hematopoietic stem cell transplantation (HSCT), although early diagnosis and treatment initiation is usually critical in order to have any significant impact on disease progression. General characteristics of MPS are indicated in the table below (adapted from Celik et al., supra, in which: HS is heparan sulfate; DS is dermatan sulfate; CS is chondroitin sulfate, e.g. chondroitin-4-sulfate or chondroitin-6-sulfate; and KS is keratan sulfate):

| | Associated | | |
|---|---|---|---|
| Type | gene | Deficient enzyme | GAG accumulation |
| MPS I | IDUA | α-L-iduronidase | HS and DS (type I-H, severe); |
| | | | DS only (types I-S and I-H/S) |
| MPS II | IDS | iduronidase-2-sulfatase | HS and DS |
| MPS IIIA | SGSH | heparan-N-sulfatase | HS |
| MPS IIIB | NAGLU | α-N-acetylglucosaminidase | HS |
| MPS IIIC | HGSNAT | α-glucosaminidase | HS |
| | | acetyltransferase | |
| MPS IIID | GNS | N-acetylglucosamine-6- | HS |
| | | sulfatase | |
| MPS IVA | GALNS | N-acetylglucosamine-6- | C6S, KS |
| | | sulfate sulfatase | |
| MPS IVB | GLB1 | β-galactosidase | KS |
| MPS VI | ARSB | N-acetylglucosamine-4- | DS, C4S |
| | | sulfatase | |
| MPS VII | GUSB | β-glucuronidase | HS, DS, C4S, C6S |
| MPS IX | HYAL1 | hyaluronidase | Hyaluronan |

Clinical diagnosis of MPS was historically made by the measurement of total GAG levels in the urine. This method is, however, impractical and costly for screening urine from newborns since collection and storage is difficult. A spot test for urine GAG levels has been developed based on dye staining, but this suffers from reliability and sensitivity problems, especially in the first few days after birth. These dye-based methods are not generally applicable to blood spot samples and are also unable to distinguish between MPS types or predict the severity of the disease with great confidence (see, e.g., Tomatsu et al., Mol Genet Metab. (2013) 110(0):42-53). Affinity binding methods (ELISA), which can detect specific GAGs such as, e.g., keratan sulfate, have been developed. Such methods can be used to assess GAG levels in blood samples, but are not suitable for the simultaneous analysis of multiple GAGs (see, e.g., Khan et al., Mol Genet Metab. (2020) 130(2):101-109). More recently, mass spectrometric methods (e.g. LC-MS/MS) have been developed to detect and quantify multiple GAGs in blood samples (see, e.g., Khaledi et al., Anal Chem. (2020) 92(17):11721-11727), although these methods are not suitable for the diagnosis of all MPS types in blood samples.

There remains, therefore, a need to improve methods for MPS diagnosis and to provide new methods for monitoring disease progression and treatment efficacy. As shown in the accompanying Examples, CD63 levels are elevated in MPS patients and may be used as a measure to detect and monitor MPS in those subjects.

Thus, in one aspect is provided a method of diagnosing MPS in a subject suspected as being at risk of suffering from MPS, the method comprising measuring the level of CD63 in a sample from the subject. A related aspect provides the use of CD63 as a biomarker in the diagnosis of MPS in a subject suspected as being at risk of suffering from MPS. Viewed in another way, these aspects provide a method for generating quantitative data for a subject, the method comprising determining the level of CD63 in a sample from the subject, wherein the subject is suspected of suffering from (or is suffering from) MPS. In embodiments, the MPS is selected from MPS I, MPS II, and MPS III. In embodiments, the MPS is selected from MPS I and MPS II. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

In embodiments, the subject is considered as being at risk of suffering from MPS through the identification of specific risk factors such as family history of the disease, genetic testing, the analysis of other characteristic biomarkers, clinical symptoms, and the like. In embodiments, the specific risk factors are selected from one or more of clinical symptoms, family history, genetic testing, enzyme activity, and GAG levels. The clinical symptoms may include one or more symptoms which affect the head and neck (e.g., macrocephaly, hearing loss, corneal clouding, and abnormal dentition), the joints and skeleton (e.g., stiffness, hip dysplasia, claw hands, and joint laxity), the cardiovasculature system (e.g., valve thickening, and left ventricular hypertrophy), the airways (e.g., recurrent respiratory infections, and obstructive airway disease), the abdomen (e.g., Hepatomegaly/splenomegaly, and umbilical/inguinal hernia), as well as neurological symptoms (e.g., developmental delay, ventriculomegaly, dilated perivascular spaces, and hyperactive or aggressive behaviour), and other symptoms (e.g., abnormal granulation in leukocytes, fetal hydrops, and proteinuria). Many of these signs and symptoms are common to different types of MPS, and patients presenting with isolated symptoms cannot typically be diagnosed with a high degree of certainty on that basis alone. Developmental delay and osteoarticular manifestations may be most relevant for screening programs that aim at the early diagnosis of these conditions (for more details of differential diagnosis of MPS see, e.g., Kubaski et al., Diagnostics (Basel). (2020) 10(3):172). Family history may include history of MPS among ancestors and/or close relatives (e.g., aunts, uncles, cousins, etc.). Genetic testing may involve sequencing some or all of the relevant gene (e.g. as shown in the table above) and comparing that with known disease-causing variants (see, e.g., Kubaski et al., 2020, supra). The mutational status of the patient may allow a diagnosis of MPS to be made with a high degree of confidence but it may not, however, be possible to correlate a variant gene with MPS absolutely. Enzyme activity may be assessed by testing for the activity of the deficient enzyme, e.g. in a whole blood or blood spot sample (see, e.g., Filocamo et al., Italian Journal of Pediatrics (2018) 44(S2):129; and Lehman et al., Rheumatology (2011) 50(S5):v41-48). GAG levels may be assessed by measuring the concentration of glycans in a sample, e.g. a whole blood, plasma, serum, or urine sample (see, e.g., Khan et al., 2020, supra).

In embodiments, the control value is a baseline CD63 level which has previously been measured in the same subject (i.e. in an earlier sample from the subject), for example measured at least 1 month prior to obtaining the test sample from the subject, e.g. at least 2, 3, 6, 9, 12, 18, or 24 months prior to obtaining the test sample from the subject. The control value may represent the CD63 level measured in a single earlier sample from the subject or it may be an average of values from multiple earlier samples. In an alternative embodiment, the control value is the CD63 level in a sample taken from one or more healthy subjects (e.g., an average value for a cohort of healthy subjects). In embodiments, the healthy subjects are matched to the subject being assessed, e.g. by age and/or sex. Typically, the control value represents the level of CD63 in the same type of sample which is being assessed in the present methods, e.g., a sample which has been obtained, processed and/or stored in the same way as the test sample. For example, where the present method utilizes a plasma sample, the control value typically represents the level of CD63 in a control plasma sample, e.g., an earlier plasma sample taken from the same subject or a plasma sample obtained from one or more healthy subjects.

In embodiments where the control value for CD63 concentration is a point value (or an average, e.g. mean, value), the CD63 level in the test sample is considered to be outside the normal range if it is at least about 5% greater than the control value, e.g. at least about 7.5%, 10%, 12.5%, 15%, 20%, 25%, 30%, 40%, 50%, 75%, or 100% greater than the control value. In some embodiments, the CD63 level in the test sample is considered to be outside the normal range if it is at least about 2 times greater than the control value, e.g. at least about 3, 4, 5, 6, 8, or 10 times the control value. In embodiments, the CD63 level in the test sample is from about 3 to about 8 times the control value, e.g. from about 3 to about 6 times the control value, or from about 4 to about 5 times the control value. In embodiments, the CD63 level in the test sample is about 4 times the control value, or about 5 times the control value. In embodiments where the control value for CD63 concentration is a range of values, e.g. described by some variability around a mean value, the CD63 level in the test sample is considered to be outside the normal range if it is more than about 1 standard error greater than the mean value, e.g. more than about 1.0, 1.1, 1.2, 1.3, 1.4, 1.5, 1.6, 1.7, 1.8, 1.9, 2.0, 2.5, or 3.0 standard errors greater than the mean value.

In embodiments, CD63 is used as the only biomarker in the methods of the disclosure.

Alternatively, measurement of the CD63 level in the sample from the subject is used alongside one or more other characteristic measures of the disease, e.g., as part of a panel of biomarkers used for diagnostic purposes. This aspect provides a method of improving the diagnosis of MPS in a subject, characterized in that CD63 is used as a biomarker. A related aspect provides the use of CD63 as a biomarker to improve a method of diagnosing MPS in a subject. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA). In embodiments, the other biomarkers used (i.e., besides CD63) do not include any of the following: α-iduronidase, α-glucosidase, saposin C, LAMP-1, LAMP-2, β-glucosidase, α-galactosidase A, iduronate-2-sulphatase, N-acetylgalactosamine 4-sulphatase, galactose 6-sulphatase, acid sphingomyelinase, galactocerebrosidase, arylsulphatase A, saposin B, heparan-N-sulphatase, α-N-acetylglucosaminidase, acetylCoA:glucosamine N-acetyltransferase, N-acetylglucosamine 6-sulphatase, β-galactosidase, β-glucuronidase, aspartylglucosaminidase, acid lipase, β-hexosamindase A, β-hexosamindase B, GM2-activator, acid ceramidase, α-L-fucosidase, α-D-mannosidase, β-D-mannosidase, neuraminidase, phosphotransferase, phosphotransferase g-subunit, palmitoyl protein thioesterase, tripeptidyl peptidase I, cathespsin K, α-galactosidase B, sialic acid transporter, CD45 leukocyte common biomolecule, or LIMP II.

In embodiments, the use of CD63 as a biomarker reduces the proportion of false positives and/or increases the proportion of true positives as compared to a corresponding diagnosis in which the CD63 level is not measured.

Another aspect provides a method of treating a subject who has been diagnosed as having MPS by a method as defined herein, the treatment comprising administering to the subject one or more therapeutic treatments for MPS. A related aspect provides a therapeutic agent for the treatment of MPS in a subject, wherein the subject has been diagnosed as having MPS by a method as defined herein. Also provided is a method for generating quantitative data for a subject, wherein the method comprises (e.g. consists of): (a) determining a level of CD63 in a sample from the subject; (b) determining whether the level of CD63 is greater than a control value (e.g., a control value measured as the CD63 level in a sample taken from one or more healthy subjects); and (c) applying one or more therapeutic treatments for MPS to the subject if the level of CD63 in the sample is greater than the control value. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

In embodiments, the therapeutic treatment or agent comprises (e.g. consists of) enzyme replacement therapy, gene therapy, and/or hematopoietic stem cell transplantation. In embodiments, the MPS is MPS I, MPS II, MPS IV, MPS VI, or MPS VII and the therapeutic treatment comprises (e.g. consists of) enzyme replacement therapy and/or hematopoietic stem cell transplantation. In embodiments, the therapeutic treatment or agent comprises (e.g. consists of) enzyme replacement therapy with α-L-iduronidase, iduronidase-2-sulfatase, heparan-N-sulfatase, α-N-acetylglucosaminidase, α-glucosaminidase, acetyltransferase, N-acetylglucosamine-6-sulfatase, N-acetylglucosamine-6-sulfate sulfatase, β-galactosidase, N-acetylglucosamine-4-sulfatase, β-glucuronidase, or hyaluronidase. In embodiments, the MPS is MPS I and the therapeutic treatment comprises (e.g. consists of) administering recombinant α-L-iduronidase (e.g. laronidase) to the subject. In other embodiments, the MPS is MPS II and the therapeutic treatment comprises (e.g. consists of) administering recombinant iduronidase-2-sulfatase (e.g. idursulfase) to the subject. In other embodiments, the MPS is MPS IVA and the therapeutic treatment comprises (e.g. consists of) administering recombinant N-acetylgalactosamine-6-sulfatase (e.g. elosulfase alfa) to the subject; the MPS is MPS VI and the therapeutic treatment comprises (e.g. consists of) administering recombinant N-acetylglucosamine-4-sulfatase (e.g. galsulfase) to the subject; or the MPS is MPS VII and the therapeutic treatment comprises (e.g. consists of) administering recombinant β-glucuronidase (e.g. vestronidase alfa) to the subject.

A further aspect provides a method for monitoring the progress of MPS in a subject diagnosed as having MPS, the method comprising: (a) measuring the level of CD63 in a first sample from the subject; (b) measuring the level of CD63 in a second sample from the subject, wherein the second sample was taken from the subject after the first sample was taken from the subject; and (c) comparing the level of CD63 in the first sample with the level of CD63 in the second sample. Typically, the method further comprises a step (d) determining that the disease is becoming more severe if the level of CD63 in the second sample is greater than the level of CD63 in the first sample, determining that the disease in the subject is not progressing if the level of CD63 in the second sample is substantially the same as the level of CD63 in the first sample, and determining that the disease is becoming less severe if the level of CD63 in the second sample is lower than the level of CD63 in the first sample. A related aspect provides the use of CD63 as a biomarker for monitoring the progress of MPS in a subject diagnosed as having MPS. Viewed in another way, these aspects provide a method for generating quantitative data for a subject diagnosed as having MPS, the method comprising the steps of: (a) determining a level of CD63 in a first sample from the subject; (b) determining a level of CD63 in a subsequent sample from the subject; and (c) comparing the level of CD63 determined in step (a) with the level determined in step (b). The method may comprise a further step (d) in which the progress of the disease is assessed based on the comparison carried out in step (c). In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

The sample may be of a type mentioned herein. In embodiments, the sample contains lysosomes and/or exosomes. In embodiments, the sample is selected from blood, a blood fraction, urine, cerebrospinal fluid, sputum, lymph, dermal tissue, renal tissue, cardiac tissue, spleen tissue, bone marrow, and the like. Most conveniently, the sample from the subject comprises (e.g. consists of) blood, a blood fraction, and/or urine. In embodiments, the sample from the subject comprises (e.g. consists of) a blood fraction selected from plasma and serum. In embodiments, the sample is a blood sample, e.g. a plasma sample. In other embodiments the sample comprises (e.g. consists of) urine. In other embodiments, the sample comprises (e.g. consists of) CSF. In embodiments, the sample does not comprise fibroblasts.

In embodiments, the first and second (or subsequent) samples are taken from the subject at an interval of at least about 1 day, e.g. at least about 2, 3, or 4 days, or at least about 1, 2, or 4 weeks. In other embodiments, the first and second (or subsequent) samples are taken from the subject at an interval of at least about 8 weeks, e.g. at least about 9, 10, 12, 15, or 20 weeks.

In embodiments, the methods comprise the ongoing measurement of one or more further samples taken from the subject, e.g., at the intervals specified above.

Another aspect provides a method for monitoring the progress of a treatment for MPS in a subject diagnosed as having MPS, the method comprising: (a) measuring the level of CD63 in a first sample from the subject; (b) administering a therapeutic treatment for MPS to the subject; and (c) measuring the level of CD63 in a second sample from the subject, wherein the second sample was taken from the subject after the therapeutic treatment was administered. Typically, the method further comprises a step (d) of determining that the treatment is successful if the level of CD63 in the second sample is lower than the level of CD63 in the first sample. In embodiments, the dosage (e.g., the amount and/or the frequency of administration) of the therapeutic treatment is to be increased if the level of CD63 determined in step (c) is substantially the same as, or greater than, the level of CD63 determined in step (a). A related aspect provides the use of CD63 as a biomarker for monitoring the progress of a treatment for MPS in a subject diagnosed as having MPS. Viewed another way, these aspects provide a method for generating quantitative data for a subject having MPS, the method comprising the steps of: (a) determining a level of CD63 in a first sample from the subject; and (b) determining a level of CD63 in a second sample from the subject after administration of a therapeutic treatment for MPS to the subject. In embodiments, the dosage of the therapeutic treatment is to be increased if the level of CD63 determined in step (b) is substantially the same as, or greater than, the level of CD63 determined in step (a). In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

In embodiments, the treatment for MPS is as described herein. Thus, the treatment may comprise (e.g. consist of) enzyme replacement therapy, gene therapy, or hematopoietic stem cell transplantation. In embodiments, the MPS is MPS I, MPS II, MPS IV, MPS VI, or MPS VII and the treatment comprises (e.g. consists of) enzyme replacement therapy and/or hematopoietic stem cell transplantation. In embodiments, the therapeutic treatment or agent comprises (e.g. consists of) enzyme replacement therapy with α-L-iduronidase, iduronidase-2-sulfatase, heparan-N-sulfatase, α-N-acetylglucosaminidase, α-glucosaminidase, acetyltransferase, N-acetylglucosamine-6-sulfatase, N-acetylglucosamine-6-sulfate sulfatase, β-galactosidase, N-acetylglucosamine-4-sulfatase, β-glucuronidase, or hyaluronidase. In embodiments, the MPS is MPS I and the treatment comprises (e.g. consists of) administering recombinant α-L-iduronidase (e.g. laronidase) to the subject. In other embodiments, the MPS is MPS II and the treatment comprises (e.g. consists of) administering recombinant iduronidase-2-sulfatase (e.g. idursulfase) to the subject. In other embodiments, the MPS is MPS IVA and the treatment comprises (e.g. consists of) administering recombinant N-acetylgalactosamine-6-sulfatase (e.g. elosulfase alfa) to the subject; the MPS is MPS VI and the treatment comprises (e.g. consists of) administering recombinant N-acetylglucosamine-4-sulfatase (e.g. galsulfase) to the subject; or the MPS is MPS VII and the treatment comprises (e.g. consists of) administering recombinant β-glucuronidase (e.g. vestronidase alfa) to the subject.

The second sample is typically taken after a period of some time has elapsed from the administration of the therapeutic treatment. In embodiments, the second sample is taken from the subject at least about 1 week after administration (e.g. after initiation) of the therapeutic treatment, e.g. at least about 2, 3, 4, 6, 8, 10, 12, 15, or 20 weeks after administration of the therapeutic treatment. In one embodiment, the second sample is taken from the subject at least about 8 weeks after initiation of the therapeutic treatment.

In embodiments, the methods comprise the ongoing measurement of one or more further samples taken from the subject, e.g., at intervals of about every 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method of treating MPS in a patient in need thereof, wherein the patient has a higher than normal blood plasma CD63 level (e.g. a level of at least about 0.25 standard deviations, such as at least about 0.5, 0.75, 1, or 1.5 standard deviations) above the mean value for a healthy population of subjects, the method comprising administering to the patient an effective amount of a therapeutic treatment for MPS. In embodiments, the healthy population of subjects is a population having a similar genetic background, e.g., a population of the same heritage and/or geographical location as the patient. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

A further aspect provides a method of treating MPS in a subject diagnosed as being at risk of developing said disease, wherein the patient has a blood plasma CD63 level which is higher than a control level in a sample previously taken from the subject (e.g. at least about 10% higher than, such as at least about 20%, 30%, 40%, 50%, 60%, 80%, or 100% higher than, the blood plasma control level), the method comprising administering to the subject an effective amount of a therapeutic treatment for MPS (e.g. as described herein). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level. In embodiments, the control value is derived from measuring the CD63 level in a single sample drawn from the subject. In other embodiments, the control value is an average is derived from measuring the CD63 level in multiple samples drawn from the subject (e.g. 2, 3, 4, 6, 8, or more samples). In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

A further aspect provides a method of reducing CD63 levels in a patient suffering from MPS (e.g. in the blood of the patient), the method comprising the step of administering an effective amount of a therapeutic treatment (e.g. an enzyme replacement therapy as described herein) to the patient. In embodiments, the reduction in CD63 is a reduction in blood plasma CD63 levels. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

Another aspect provides a method of treating or preventing the development or progression of MPS in a subject assessed as being at risk of suffering from MPS, the method comprising the steps of: (a) taking a first biological sample (e.g. blood or a blood fraction such as plasma) from the subject and analyzing said sample for CD63 concentration; (b) if the CD63 concentration is above a control value, starting the subject on a course of therapeutic treatment (e.g., a therapeutically effective amount of an enzyme replacement therapy, such as those described herein), and optionally: (c) after treating the subject, taking a second biological sample (e.g. being the same type as the first biological sample) from the subject and analyzing said sample for CD63 concentration to determine a change in CD63 level; and (d) adjusting the therapeutic treatment based on the change in CD63 level observed. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

In embodiments, the control value is about 10% higher (e.g. about 20%, 30%, 40%, 50%, 60%, 80%, or 100% higher than) than a control level in a sample previously taken from the same subject. In embodiments, the control level is derived from measuring the CD63 level in a single sample drawn from the subject. In other embodiments, the control value is an average is derived from measuring the CD63 level in multiple samples drawn from the subject (e.g. 2, 3, 4, 6, 8, or more samples). In embodiments, the blood plasma control level is from a sample drawn from the subject between about 1 and about 52 weeks (e.g. between 4 and 32 weeks, or between 12 and 26 weeks, such as about 12, 16, 22, 26, or 30 weeks) prior to the measurement of said blood plasma CD63 level.

In embodiments, the treatment for MPS is as described herein. Thus, the treatment may comprise (e.g. consist of) enzyme replacement therapy, gene therapy, and/or hematopoietic stem cell transplantation. In embodiments, the treatment comprises (e.g. consists of) enzyme replacement therapy with α-L-iduronidase, iduronidase-2-sulfatase, heparan-N-sulfatase, α-N-acetylglucosaminidase, α-glucosaminidase, acetyltransferase, N-acetylglucosamine-6-sulfatase, N-acetylglucosamine-6-sulfate sulfatase, β-galactosidase, N-acetylglucosamine-4-sulfatase, β-glucuronidase, or hyaluronidase.

The second sample is typically taken after a period of time has elapsed from the administration of the therapeutic treatment. In embodiments, the second sample is taken from the subject at least about 1 week after administration (e.g., after initiation) of the therapeutic treatment, e.g. at least about 2, 3, 4, 6, 8, 10, 12, 15, or 20 weeks after administration of the therapeutic treatment. In one embodiment, the second sample is taken from the subject at least about 8 weeks after initiation of the therapeutic treatment.

In embodiments, the methods comprise the ongoing measurement of one or more further samples taken from the subject, e.g., at intervals of about every about 1 week, such as about every 2, 4, 6, 8, 10, or 12 weeks. This ongoing measurement can be particularly valuable in the case where the dosage of the therapy is being adjusted based on the change in CD63 level in the subject.

Another aspect provides a method for adjusting the dosage of a therapeutic treatment for MPS in a subject receiving said therapeutic treatment, the method comprising: (a) measuring the level of CD63 in a first sample from the subject; (b) measuring the level of CD63 in a second sample from the subject, wherein the second sample is taken from the subject after administration of one or more doses of the therapeutic treatment; and (c) adjusting the dosage of the therapeutic treatment based on the difference between the level of CD63 in the first sample and the level of CD63 in the second sample. Typically, the dosage adjustment in step (c) comprises maintaining the dosage of the therapeutic treatment if the level of CD63 in the second sample is lower than the level of CD63 in the first sample and increasing the dosage (e.g., the amount and/or the frequency of administration) of the therapeutic treatment if the level of CD63 in the second sample is substantially the same as, or greater than, the level of CD63 in the first sample. In embodiments, the dosage (e.g., the amount and/or the frequency of administration) of the therapeutic treatment is decreased if the level of CD63 in the second sample is lower than the level of CD63 in the first sample. A related aspect provides the use of CD63 as a biomarker for adjusting the dosage of a therapeutic treatment for MPS in a subject receiving said therapeutic treatment. In embodiments, the MPS is selected from MPS I, MPS II, and MPS III. In embodiments, the MPS is MPS I (e.g. type I-H, I-S, or I-H/S). In other embodiments, the MPS is MPS II. In other embodiments, the MPS is MPS III (e.g. type IIIA, IIIB, IIIC, or IIID). In embodiments, the MPS is not MPS I. In embodiments, the MPS is not MPS III (e.g. MPS IIIA). In embodiments, the MPS is not MPS I or MPS III (e.g. MPS IIIA).

### Assays

Assays which can be used to measure and quantify CD63 levels in biological samples are known. Specific examples of suitable assays are given in the Examples which follow. Generally speaking, the assays rely on having a molecule which binds specifically to CD63 (e.g. an antibody) and which can itself be detected and/or quantified, either directly or via another binding partner (e.g. using a labelled universal antibody).

Thus, in embodiments, CD63 detection and/or quantification is performed by immunoassay (e.g. ELISA, microfluidic ELISA, or bead-based high sensitivity ELISA, or Proximity Extension Assay), mass spectroscopy, microchip, biophysical assay (e.g. surface plasmon resonance using anti-CD63 capture antibodies, such as from Biocore®), nanoneedle bioarray (e.g. from Nanomosaic®), or nucleic acid binding aptamers (e.g. from Somalogic®). An exemplary embodiment employs an ELISA, for example an indirect or sandwich ELISA in which: the primary or capture antibody binds specifically to CD63 (e.g. a mouse antibody binding to human CD63); and the secondary or detection antibody binds to the first antibody (e.g. a goat anti-mouse IgG), or to a different portion of CD63, and is labelled for detection and/or quantification. The primary or capture antibodies are typically monoclonal antibodies which may react with CD63 from one species or from more than one species. Such antibodies are widely available from commercial sources (for example Invitrogen®, AbCam®, etc.). Moreover, the sequence of CD63 is well known and the skilled person could produce monoclonal antibodies to CD63 using standard techniques (See also “Leukocyte and Stromal Cell Molecules: The CD Markers”, Wiley; Ed. Zola et al., 2007, pp. 150). Another exemplary embodiment employs cell sorting techniques, e.g. FACS using fluorescently labelled antibodies to CD63, CD8, and/or CD81.

An alternative exemplary embodiment employs a Proximity Extension Assay, which uses a matched pair of CD63-binding antibodies, wherein each of the matched pair of antibodies is labelled with a unique oligonucleotide such that the oligonucleotides hybridize when the antibodies bind to their target. The annealing product is then amplified by PCR and detected (e.g., in a multiplexed fashion), typically in a high throughput fluidic chip system.

Samples for use in accordance with the present methods may, for example, be purified and/or separated from other (e.g. non CD63-containing) components in the sample. Samples may also be concentrated for component of interest, e.g. exosomes. Methods for the concentration and/or purification of exosomes and other CD63-containing components are known in the art and/or are described herein. By way of example, samples may be enriched for exosomes by ultracentrifugation and/or antibody capture methods. Ultracentrifugation of plasma, serum, urine, or cell culture samples may include, for example, centrifugation at 100,000 g to pellet exosomes and resuspension in an appropriate buffer. Affinity capture methods may, for example, use antibody-coated beads to capture an exosome membrane target, followed by recovery of the beads by centrifugation or the use of magnets.

### Kits

A further aspect provides a kit for (e.g. suitable for) detecting or diagnosing lysosomal dysfunction or aberrant glycosphingolipid processing in a sample from a subject, the kit comprising means for detecting CD63 and optionally means for detecting one or more further biomarkers of lysosomal dysfunction. In embodiments, the means for detecting CD63 comprises an anti-CD63 antibody (e.g. a monoclonal antibody). In embodiments, the means for detecting CD63 comprises a pair of anti-CD63 antibodies, each of which is linked to a unique oligonucleotide, whereby the unique oligonucleotides can hybridize when the pair of antibodies bind to CD63.

Another aspect provides a kit for (e.g. suitable for) monitoring the progression of a specific lysosomal storage disease or monitoring the response of a specific lysosomal storage disease to treatment in a subject, the kit comprising means for detecting CD63 and optionally means for detecting one or more further biomarkers of lysosomal dysfunction. In embodiments, the means for detecting CD63 comprises an anti-CD63 antibody (e.g. a monoclonal antibody). In embodiments, the means for detecting CD63 comprises a pair of anti-CD63 antibodies, each of which is linked to a unique oligonucleotide, whereby the unique oligonucleotides can hybridize when the pair of antibodies bind to CD63.

A yet further aspect provides a kit for (e.g. suitable for) detecting or diagnosing a specific lysosomal storage disease in a subject, the kit comprising: (a) means for detecting CD63 in a sample from the subject; and (b) means for detecting one or more biomarkers of said lysosomal storage disease in a sample from the subject. In embodiments, the means for detecting CD63 comprises an anti-CD63 antibody (e.g. a monoclonal antibody). In embodiments, the means for detecting CD63 comprises a pair of anti-CD63 antibodies, each of which is linked to a unique oligonucleotide, whereby the unique oligonucleotides can hybridize when the pair of antibodies bind to CD63.

In embodiments, the lysosomal storage disease is Fabry disease and the kit comprises: (a) means for detecting CD63 in a sample from the subject (e.g. an anti-CD63 antibody, or a matched pair of anti-CD63 antibodies, each of which is linked to a unique oligonucleotide); and (b) means for detecting one or more biomarkers of Fabry disease in the sample (e.g. means for detecting GL3 and/or lyso-GL3 in the sample).

In embodiments, the lysosomal storage disease is Gaucher disease and the kit comprises: (a) means for detecting CD63 in a sample from the subject (e.g. an anti-CD63 antibody, or a matched pair of anti-CD63 antibodies, each of which is linked to a unique oligonucleotide); and (b) means for detecting one or more biomarkers of Gaucher disease in the sample (e.g. means for detecting lyso-GL1 in the sample).

In embodiments, the lysosomal storage disease is MPS (e.g. MPS I, MPS II, or MPS III) and the kit comprises: (a) means for detecting CD63 in a sample from the subject (e.g. an anti-CD63 antibody, or a matched pair of anti-CD63 antibodies, each of which is linked to a unique oligonucleotide); and (b) means for detecting one or more biomarkers of said MPS in the sample, such as means for detecting dermatan sulfate and optionally also heparan sulfate in the sample (e.g. in the case of MPS I), means for detecting dermatan sulfate and heparan sulfate in the sample (e.g. in the case of MPS II), or means for detecting heparan sulfate in the sample (e.g. in the case of MPS III).

The kits of the disclosure may further comprise instructions for use of the kit in one or more of the methods described herein, e.g. in one or more methods for the detection and/or diagnosis of a lysosomal storage disease, lysosomal dysfunction, or aberrant glycosphingolipid processing in a biological sample.

Having been generally described herein, the follow non-limiting examples are provided to further illustrate this disclosure.

### Examples

### Experimental Protocols

### Assays for Proteins

CD63 was measured by Olink® profiling, as described below, using the Olink® Target96 Neuro-Exploratory panel (Olink Proteomics AB, Uppsala, Sweden) according to the manufacturer's instructions.

Chitotriosidase activity was assayed according to the methods described in Schoonhoven, et al., Clin Chim Acta. (2007) 381(2):136-139, with minor modifications. Briefly, 5 μL of serum sample was mixed with 100 μL of 26 μM 4-methylumbelliferyl-β-D-N,N′,N″-triacetylchitotrioside (Sigma, M5639) in 0.1 M/0.2 M citrate-phosphate buffer. This mixture was incubated at 37° C. for 15 min. To stop the reaction, glycine-sodium hydroxide buffer was added (210 μL of 0.5 M Gly-NaOH, pH 10.6). Fluorescence was measured at 360 nm excitation and 455 nm emission using a plate reader (Biotek®). Chitotriosidase activity can also be assayed according to the methods described in Adelino et al., JIMD Rep. (2013) 9:85-91, and reported in units of nmol/hr/ml.

CCL18 levels were measured by a sandwich ELISA according to the method described in Boot, et al., Blood (2004) 103(1):33-39.

Protein detection can be performed using Olink® (Olink Proteomics AB, Uppsala, Sweden) profiling in accordance with the manufacturer's recommended protocols (for exemplary methods, see, e.g., Assarsson et al., PLoS ONE (2014) 9(4):e95192; and Jabbari et al., Journal of Neurology, Neurosurgery & Psychiatry (2019) 90:768-773). The Proximity Extension Assay (PEA) technology used for the Olink® protocol has been well described (Assarsson et al., 2014, supra), and enables 92 analytes per panel to be analysed simultaneously, using 1 μL of each sample. In brief, pairs of oligonucleotide-labelled antibody probes bind to their targeted protein, and if the two probes are brought in close proximity the oligonucleotides will hybridize in a pair-wise manner. The addition of a DNA polymerase leads to a proximity-dependent DNA polymerization event, generating a unique PCR target sequence. The resulting DNA sequence can subsequently be detected and quantified using a microfluidic real-time PCR instrument (Biomark HD, Fluidigm). Data is then quality controlled and normalized using an internal extension control and an inter-plate control, to adjust for intra- and inter-run variation. The final assay read-out is presented in Normalized Protein expression (NPX) values, which is an arbitrary unit on a log 2-scale where a high value corresponds to a higher protein expression. All assay validation data (e.g., detection limits, intra- and inter-assay precision data, etc.), experimental protocols, and data processing are available on the manufacturer's website (www.olink.com).

The PEA assay can be calibrated for quantitation of protein level, e.g. of CD63. Typically, this involves preparation of samples of known concentration (e.g., by titrated dilution in a buffer as defined above) and the generation of an absolute concentration calibration curve. Recombinant human CD63 is typically obtained (e.g., from a commercial supplier) which has been produced in a mammalian expression system to promote correct folding and/or post-translational modification (e.g., glycosylation), although proteins may be expressed in baculovirus or E. coli systems using known techniques. The concentration may be determined using well-known methods, for example using the bicinchoninic acid (BCA) assay with BSA samples of known concentration. Concentrations may also be determined by measuring total amino acid concentration after hydrolysis of the protein. A suitable range of decreasing protein concentrations is chosen to be within the dynamic range of the Olink® assay. For example, 3-fold dilutions may be used to yield samples having a protein concentration of 1, 3, 9, 12, 36, 108, 324, and 972 pg/ml. A 0 pg/ml sample (i.e. just buffer) is used as a blank to determine the background. Once calibrated using samples of known CD63 concentration, the assay is used to test human patient samples in the same assay experiment. Human patient samples are typically run undiluted but may be diluted by known amounts of buffer where the protein level falls outside the range of the calibration curve. The absolute concentration of CD63 in the test samples can be calculated by reference to the calibration curve—by comparison of Olink® NPX values (for example the triplicate determination average)—by fitting the calibration sample values to either a linear fit against pg/ml, or by curve fitting such as 4 parameter non-linear curve fitting, as appropriate to give a good curve fit. Provided the test sample NPX values are within the dynamic range of the calibration curve—e.g., being above the level of the 0 pg/ml blank with acceptable variability (e.g. within 20% CV)—the absolute concentration is accurately determined by reference to the fitted calibration curve. In this way, the PEA assay can provide absolute quantitation (in pg/ml) of protein level.

For the measurement of protein levels in CSF, the following protocol was employed: Biomarker expression in CSF was measured using four Olink® Explore panels (Cardiometabolic, Inflammation, Neurology, and Oncology), as described, e.g., by Wik et al. (“Proximity Extension Assay in Combination with NextGeneration Sequencing for High-throughput Proteome-wide Analysis, 2021, Mol Cell Proteomics 20, 100168). Briefly, 10 μL of CSF samples and Olink controls were placed into a 384-well Sample Source Plate and then diluted 1:10, 1:100, 1:1000 and 1:100,000 in 384-well Sample Diluent Plates using Mosquito and Dragonfly liquid handlers (both from SPT Labtech). For immunoreaction, these diluted samples were mixed with the Olink® probes (DNA oligo-conjugated antibodies) in 384-well Incubation Plates using the Mosquito handler and incubated at 4° C. for 16-24 hours. The following day, reagent mixtures for the first PCR amplification step (PCR1 step) were added to the immunoreaction mixtures using the Dragonfly handler, and PCR1 reaction for the pre-amplification step was conducted using ProFlex PCR System (Applied Biosciences). PCR1 products were pooled using epMotion 5075 liquid handler (Eppendorf), then the reagents for the second PCR amplification step (PCR2 step) were added using the same handler and PCR2 reaction for amplification and sample indexing was conducted using the ProFlex System. PCR2 products were pooled using the epMotion handler to create four libraries for each Olink® Explore panel, which were then purified using magnetic beads and their QC was performed using Bioanalyzer 2100 (Agilent). The four libraries were analyzed by Next Generation Sequencing using NovaSeq 6000 (Illumina). Analytes with more than 10% missing data were excluded from the analysis. No imputation for missing data was performed.

Alternative methods may be used to measure protein levels, such as CD63, including for example immunoassays using antibodies to CD63 (e.g., ELISAs which are widely commercially available, microfluidic ELISAs such as Protein Simple Ella, or bead based high sensitivity ELISAs such as SIMOA Quanterix), biophysical methods using anti-CD63 antibodies to capture CD63 (e.g. Biacore surface plasmon resonance), and nano needles (e.g. Nanomosaic), or nucleic acid binding aptamers (e.g. Somalogic). Plasma, serum, cell, and tissue samples for traditional ELISA assays are typically diluted in sample dilution buffer by 4× or more to give acceptable assay performance (dilutional linearity).

### Assays for Glycosphingolipids

Glycosphingolipids (lyso-GL1, GL3, and lyso-GL3) were measured by ESI-LC-MS/MS according to the method described in Murugesan et al., Am J Hematol. (2016) 91(11):1082-1089. In brief, 20 μL aliquots of plasma were added to 1 mL of chloroform:methanol (2:3) in an Eppendorf tube, mixed and then centrifuged. Supernatant was removed and extracted with chloroform (220 μL) and water (520 μL) by mixing and centrifugation. The upper phase was re-extracted with chloroform and added to the lower phase. The combined sample was dried, resuspended in 100 μL methanol:water (9:1) and injected into an LC-MS/MS system for tandem mass spectroscopic analysis. Separation of glycosphingolipids and other matrix components was achieved using UPLC under gradient conditions with two mobile phases: 0.1% formic acid in water; and 0.1% formic acid in acetonitrile. Mass spectrometry (MS) was performed in select ion monitoring mode, typically using the parent (M+H⁺) ion transition (e.g. m/z 462.5>282.4 for lyso-GL1). Assays were calibrated using known glycosphingolipid standards (dimethylpsychosine, 13C6-labelled lyso-GL3 (GelbChem), and C17-GL3 (Matreya LLC, cat no. 1523) as internal standards for lyso-GL1, lyso-GL3, and GL3 respectively).

### Sample Preparation

Plasma and serum sample preparation followed standard protocols. Cell samples were prepared by mixing/agitation. Tissue samples were solubilized by homogenization in a standard buffer (e.g. PBS; or 50 mM Tris/HCl pH 7.5, optionally containing a mild detergent such as 0.1% TWEEN20 and/or a carrier protein such as 1% BSA, which may improve stability and/or reproducibility). Additional detergent, e.g. up to 1% Triton, was typically added to the tissue samples at higher concentrations to aid solubilisation. Plasma and serum samples were typically run undiluted in the Olink® assays. Preparation of cell and tissue samples involving addition of buffer to a final concentration of protein within the dynamic range of the Olink® assay.

Samples to be tested may be enriched for exosomes, e.g. using FACS as described herein. Alternative methods for exosome enrichment or purification include ultracentrifugation and antibody capture methods, both of which are known and readily available. A standard ultracentrifugation for preparing exosomes from plasma/serum, urine, or cell cultures would include, for example centrifugation at 100,000 g for 1 hour in a typical floor standing ultracentrifuge using typical rotors and buckets that hold a 10 ml-50 ml sample, or for 5 minutes in a bench-top ultracentrifuge using 0.5 ml or 1.5 ml sample tubes. Exosomes pellet to the bottom of the tube and are resuspended in an appropriate buffer. Commercial kits are available (e.g. from System Biosciences Inc.) for antibody affinity capture of exosomes and exosome surface/membrane proteins. The methods typically use antibody-coated beads to capture the exosome target, followed by recovery of the beads by centrifugation or the use of magnets.

### Analysis of Patient and Control Samples for Biomarkers of LSDs

Plasma from Fabry Patients and Normal Controls

Plasma from patients with FD was acquired from 3 different cohorts—hereafter referred to as (i) ACT/LTS, (ii) Cohort 1, and (iii) Cohort 2.

The ACT/LTS samples were taken from the Sanofi-Venglustat Phase II trial. This trial consisted only of male patients. Their demographic information is given in Table 1 below. The Sanofi-Venglustat Phase II trial was a longitudinal study, and patient plasma was collected at 6 set intervals: Baseline (0 weeks), 12 weeks, 26 weeks, 52 weeks, 104.2 weeks, and 156.4 weeks. There are missing timepoints for some patients. During this trial, all patients were treated with venglustat (a substrate reduction therapy-SRT).

Cohort 1 is a collection of plasma samples provided by Dr. Gavin Oudit from the University of Alberta. The patient demographics are given in Table 1 below. Briefly, these plasma samples were from a mix of male and female patients with ages ranging from 25 to 68. They were non-longitudinal samples (e.g. 1 sample per patient), and the plasma samples were a mix of treated Fabry patients (enzyme replacement therapy-ERT), untreated Fabry patients, and healthy control subjects.

Cohort 2 is a collection of plasma samples provided by Dr. Michael Mauer from the University of Minnesota. The patient demographics are given in Table 1 below. Briefly, these plasma samples were from a mix of male and female patients with ages ranging from 4 to 59. They were mostly non-longitudinal samples (e.g. <3 samples per patient). There were some longitudinal patients with baseline, and some without. The plasma samples were from a mix of treated (enzyme replacement therapy-ERT) and untreated Fabry patients.

Healthy controls for the Fabry plasma samples were mostly from commercial sources (Sanguine Bioscience and BioIVT). These plasma samples were age and sex matched with the Fabry cohort and their demographics are given in Table 1 below. Additional healthy controls were provided with the Cohort 1 samples.

TABLE 1
Patient demographics for the Fabry disease cohorts

| | Fabry disease samples | | | | Healthy control samples | | | |
|---|---|---|---|---|---|---|---|---|
| | | Cohort 1 | Cohort | | | | Cohort 1 | |
| Cohorts | ACT/LTS | (diseased) | 2 | Total | BioIVT | Sanguine | (healthy) | Total |
| Number of | 44 | 23 | 48 | 115 | 28 | 35 | 12 | 75 |
| Samples (n) | | | | | | | | |
| Age (mean) | 29 | 42 | 32 | | 27 | 40 | 42 | |
| Age (range) | 18 to 40 | 25 to 68 | 4 to 59 | 4 to 68 | 19 to 35 | 18 to 64 | 24 to 61 | 18 to 64 |
| Male | 9 | 13 | 14 | 36 | 28 | 15 | 7 | 50 |
| patient | | | | | | | | |
| count | | | | | | | | |
| Female | 0 | 10 | 13 | 23 | 0 | 20 | 5 | 25 |
| patient | | | | | | | | |
| count | | | | | | | | |
| Untreated | 9 | 7 | 30 | 46 | | | | |
| samples | | | | | | | | |
| (baseline) | | | | | | | | |
| Treated | 35 | 16 | 25 | 76 | | | | |
| samples | | | | | | | | |
| ERT | 0 | 16 | 25 | 41 | | | | |
| SRT | 35 | 0 | 0 | 35 | | | | |
| Longitudinal | 9 | 0 | 2 | 11 | | | | |
| with | | | | | | | | |
| baseline | | | | | | | | |
| Longitudinal | 0 | 0 | 2 | 2 | | | | |
| without | | | | | | | | |
| baseline | | | | | | | | |

Serum from Gaucher Patients and Normal Controls

Samples from the Gaucher cohort came from Yale University (Prof Pram Mistry). The patient demographics are given in Table 2 below. Briefly, the Gaucher cohort consisted of 89 samples from 21 patients, both male and female. These samples spanned a large age range, with an average age of 50.5 years. Only 2 of the patients had baseline samples, but 15 patients had longitudinal data. Most of the longitudinal data were from samples taken years after the initial treatment. Most patients were treated with ERT, but some changed to SRT at some point during treatment.

The healthy control samples came from commercial (BioIVT) and internal (Genzyme donor program samples, non-clinical and internal clinical studies) sources. Patient demographics are given in Table 2 below. Briefly, there were 43 samples for 43 healthy donors; there were no longitudinal healthy control samples. The average age of healthy control patients for the Gaucher cohort was 45 years, and the age range broadly covered a similar range as that of the diseased samples.

TABLE 2
Patient demographics for the Gaucher disease cohort

| | Gaucher | |
|---|---|---|
| Cohorts | disease samples | Control samples |
| Number of Samples (n) | 89 | 43 |
| Age (mean) | 50.5 | 45 |
| Age (range) | 5 to 83 | 24 to 76 |
| Male patient count | 8 | 14 |
| Female patient count | 13 | 29 |
| Untreated samples (baseline) | 2 | |
| Treated samples | 87 | |
| ERT | 20 | |
| SRT | 5 | |
| Longitudinal with baseline | 2 | |
| Longitudinal without baseline | 13 | |

Plasma from MPS Patients and Normal Controls

Patient demographics for the MPS cohort (samples provided by Dr. Giugliani-Univ of Porto Alegre) are given in Table 3 below. Briefly, patients were all young, with an average age of about 6 years. There were 28 samples for 28 patients; there were no longitudinal samples in this cohort. Samples were from a mix of male and female subjects. Furthermore, none of the patients were on treatment, so they were all baseline disease samples. Patient diseases covered MPS I, MPS II, and MPS IIIA, with near equal representation.

Patient demographics in the healthy control cohort (samples from BioIVT) are given in Table 3 below. Patients in the healthy control cohort were age-matched to the disease patients; they were all pediatric samples with an average age of about 6 years. In total there were 19 healthy control samples from 19 patients—a mix of both male and female donors.

TABLE 3
Patient demographics for the MPS cohort

| Cohort | MPS samples | Control samples |
|---|---|---|
| Number of Samples (n) | 28 | 19 |
| Age (mean) | 5.9 | 6.3 |
| Age (range) | 0.75 to 26 | 1 to 12 |
| Male patient count | 16 | 8 |
| Female patient count | 12 | 11 |
| Untreated samples (baseline) | 28 | |
| Treated samples | 0 | |

CSF from MPS and Gaucher Disease (Type 3) Patients

While serum biomarkers can provide insights into systemic changes, the analysis of cerebrospinal fluid (CSF) biomarkers may offer a direct and more accurate assessment of neurological processes occurring within the CNS. Therefore, the potential of CD63 as a CSF biomarker for detecting MPS and for detecting GD3 and evaluating treatment response in GD3 was investigated.

CSF samples were meticulously collected from consenting patients at each center in accordance with center-specific protocols to ensure consistency and reliability. To minimize batch effects, the samples were randomized during plating based on center, sex, and disease status. The Olink proteomics method (above) was employed to profile the changes in protein biomarkers within the CSF samples.

Patient demographics for the MPS cohort are given in Table 4 below. As our data suggested no significant age-related difference for CD63, healthy adult CSF samples served as reference controls, allowing for comparative analysis. Controls were not age-matched (pediatric CSF control samples are not readily available).

TABLE 4
Patient demographics for the MPS/CSF cohort

| Group (n) | Sex (M/F) | Age (yr) |
|---|---|---|
| Control (40) | 19/21 | 20-60 |
| MPS I (17) | 11/6 | 2~15, 19, 29, 30 |
| MPS II (11) | 11/0 | 2~16, 21, 29 |

Patient samples for the GD3 study were collected as part of the Sanofi-sponsored LEAP trial, with the informed consent of patients (see, e.g., Schiffmann et al., Brain (2023) 146(2):461-474 for details). Samples from 11 adult patients were obtained through lumbar puncture at baseline and Weeks 26 and 52 when the patients were receiving a combination treatment of venglustat and imiglucerase (Cerezyme®, Sanofi, Cambridge USA). Healthy adult CSF samples were used as controls. The Olink proteomics method (above) was employed to analyze the protein biomarker changes within the CSF samples.

### RESULTS AND CONCLUSIONS

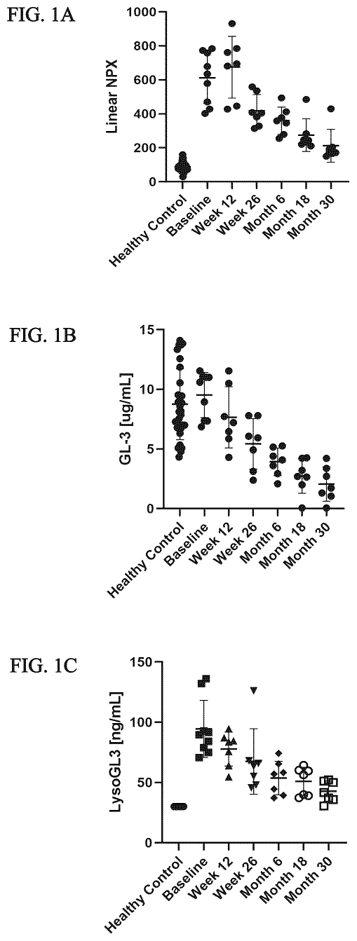

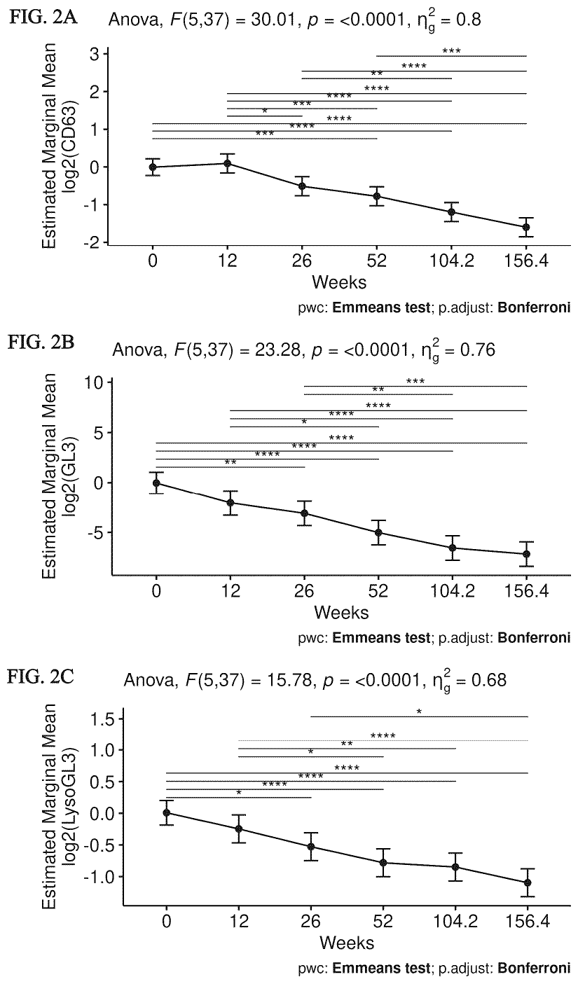

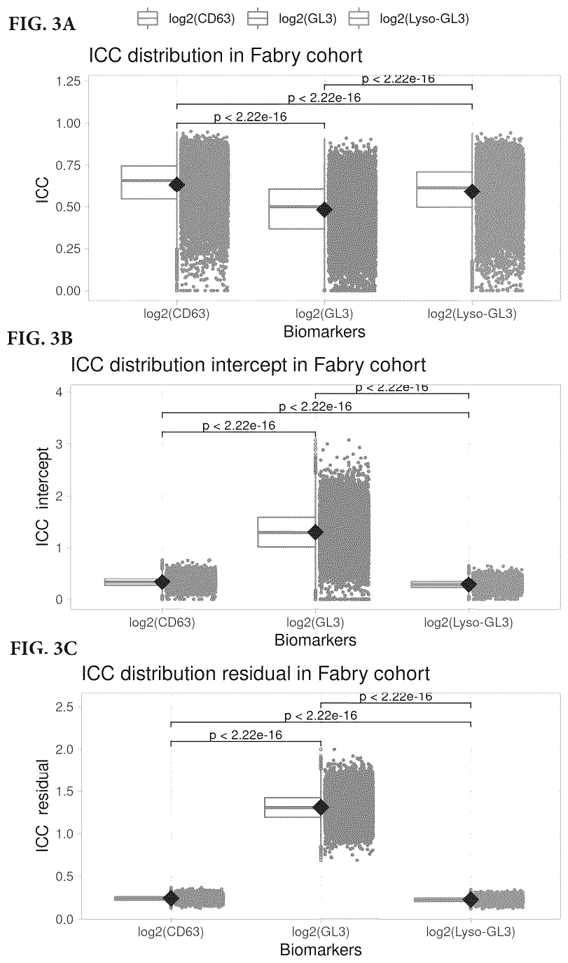

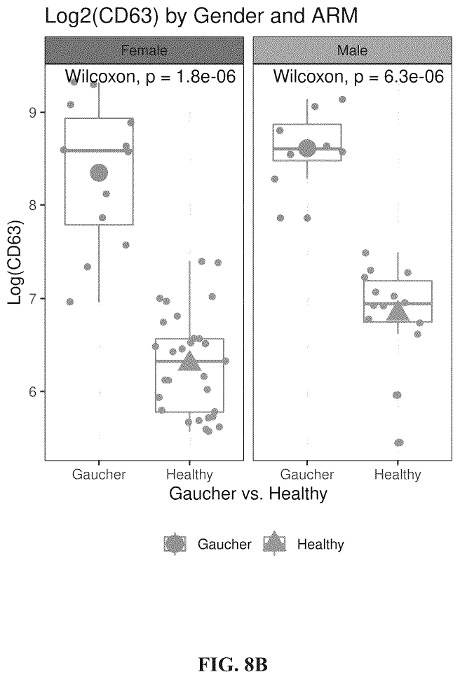

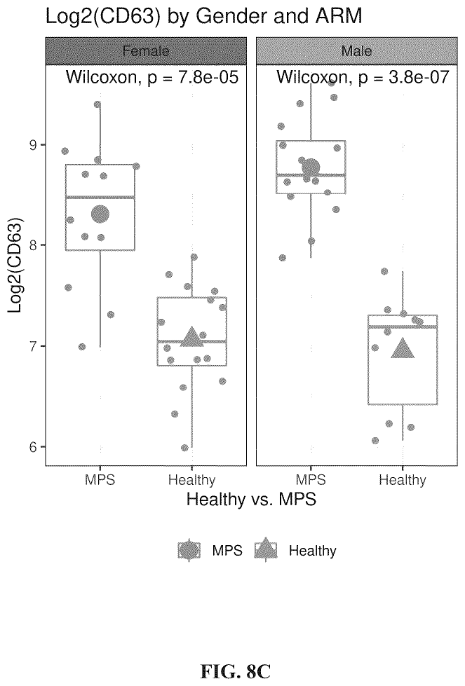

The results of the assays carried out on the Fabry samples demonstrate that CD63 is significantly upregulated in the plasma of untreated Fabry patients (“Baseline” in the longitudinal study shown in FIG. 1A). On treatment, CD63 levels decrease over a period of 30 months towards a healthy level (FIG. 1A; “Linear NPX”). Response to treatment is observed after 12 weeks and the subsequent decrease in CD63 levels is consistent across several years (FIG. 2; showing a linear drop in estimated marginal mean (log 2) values for CD63 from 12 to 156.4 weeks). The response of CD63 to venglustat treatment is comparable to canonical Fabry biomarkers GL3 and lyso-GL3 in terms of its between-patient variability (FIG. 3A). Within-patient variability is much lower for CD63 and lyso-GL3 than for GL3 (FIG. 3C) When analysed separately, the CD63 response appears to be greater in male Fabry patients than in female patients (FIG. 8A).

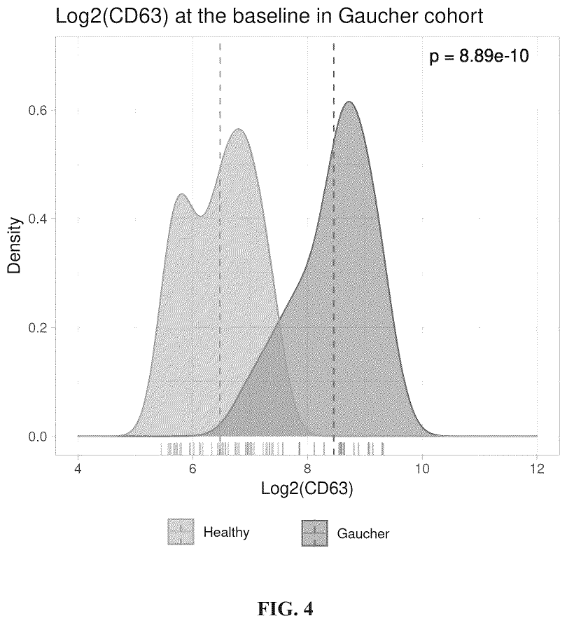

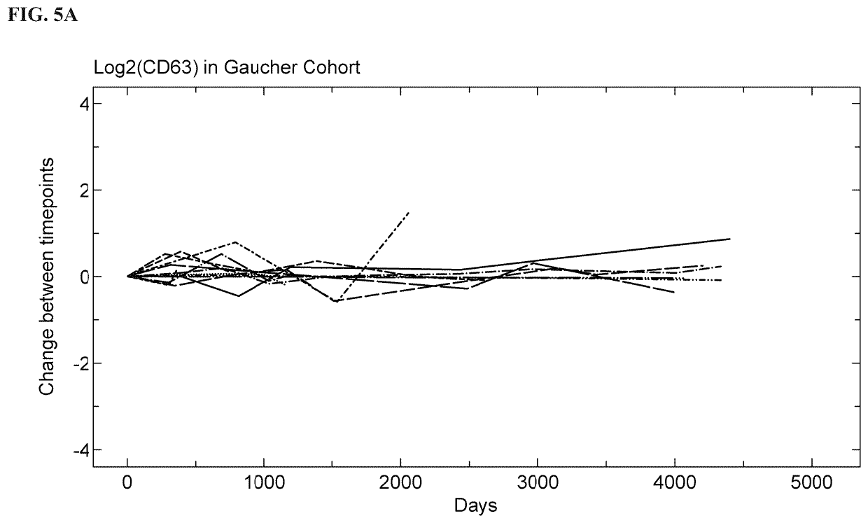

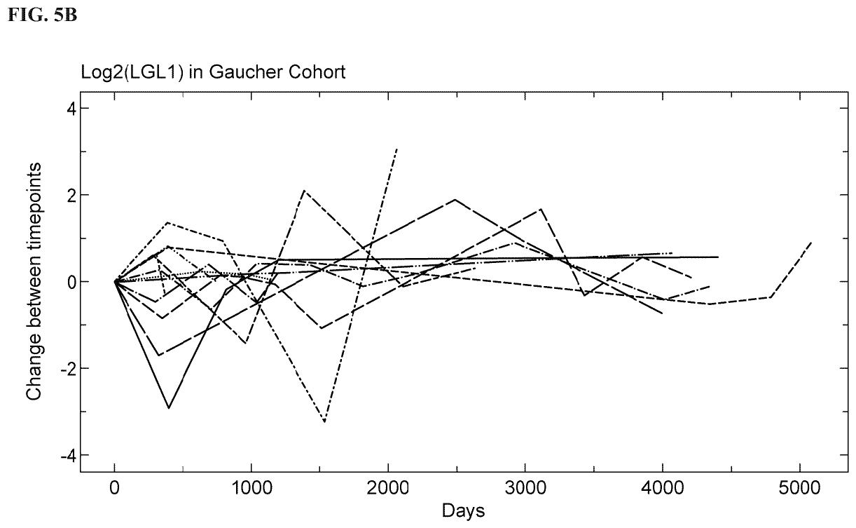

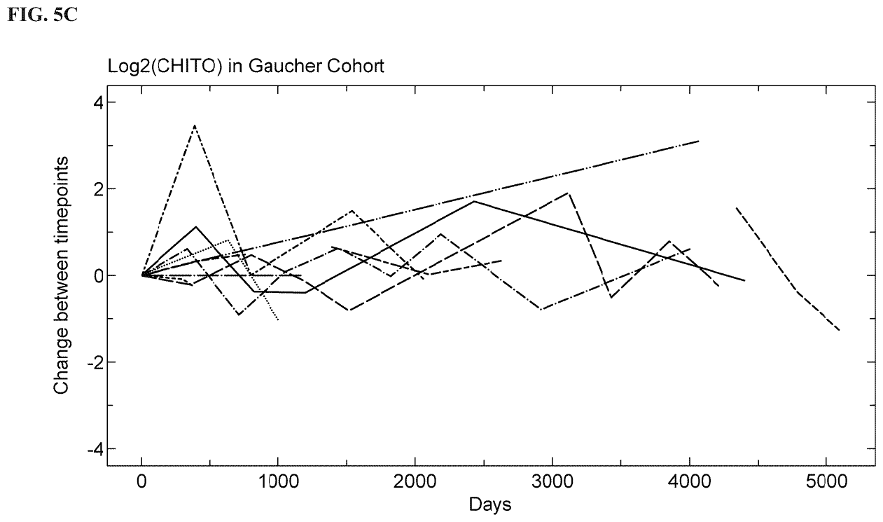

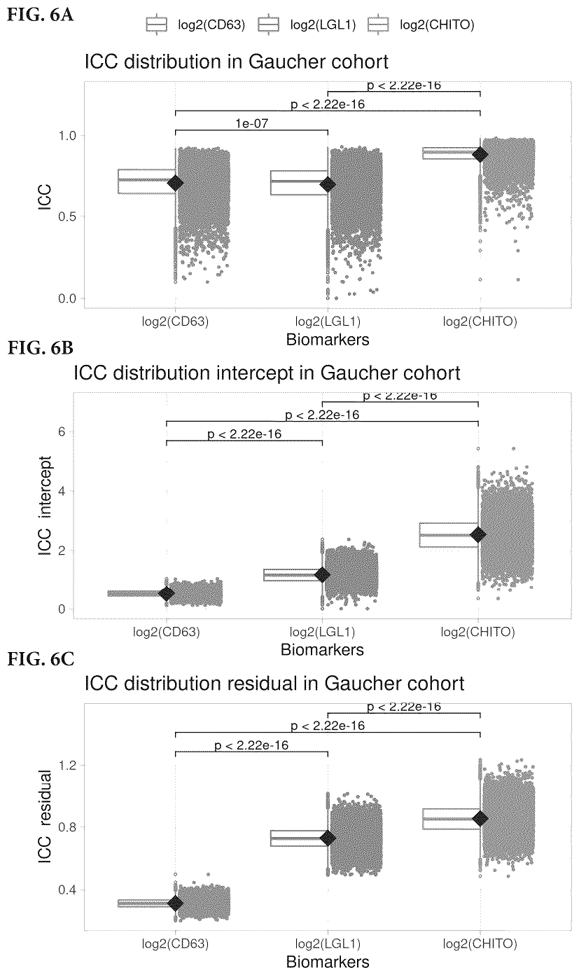

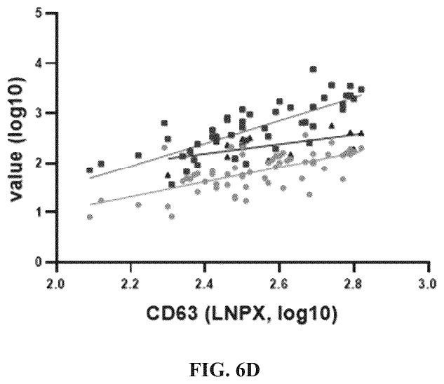

Analysis of the samples from the Gaucher cohort demonstrates that CD63 is also significantly upregulated in the plasma of Gaucher patients as compared to healthy controls (FIG. 4). There appears to be little difference in the level of CD63 in the samples from male Gaucher patients as compared to female Gaucher patients (FIG. 8B). The day-to-day variability of CD63 levels in Gaucher patients undergoing long-term treatment (FIG. 5A) is lower than the variability of other established biomarkers, such as lyso-GL1 (FIG. 5B) or chitotriosidase (FIG. 5C). This low level of within-patient variability is highly significant (FIG. 6C; p<0.002) and establishes CD63 as an exceptional biomarker for monitoring LSD status and progression, particularly as compared with the known biomarkers lyso-GL1 and chitotriosidase. Moreover, CD63 levels correlate with levels of known biomarkers for Gaucher disease (FIG. 6D).

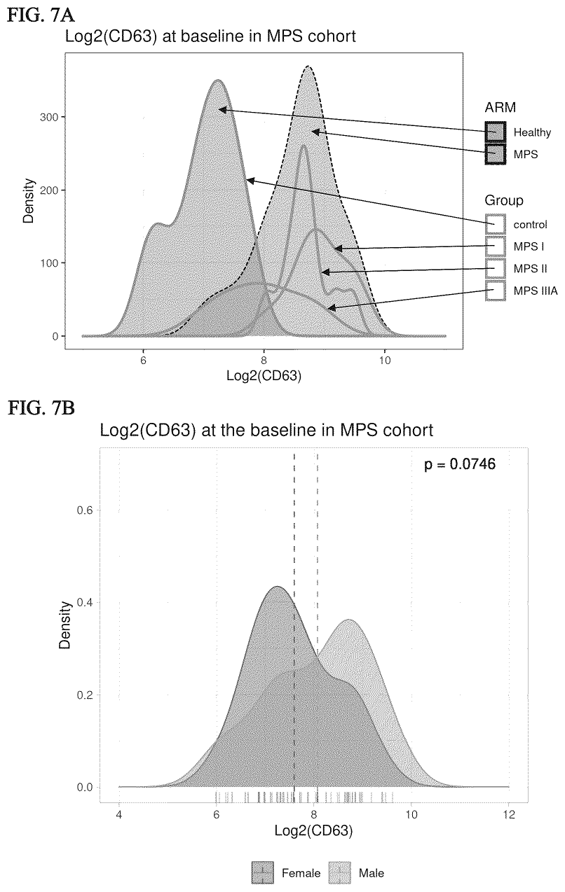

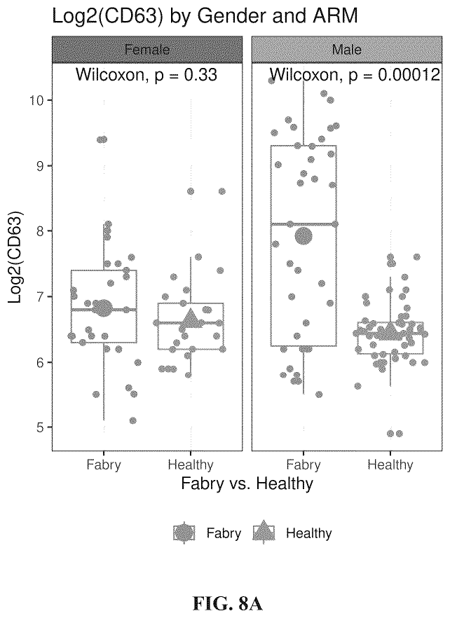

CD63 levels are also significantly elevated in MPS patients (FIG. 7A), both male and female (FIG. 8C). There does not appear to be a statistically significant distinction between average CD63 levels in male and female patients, although the different profiles of the distributions may suggest some differences in sub-types for each population (FIG. 7B, p>0.05; see also FIG. 8C). This observation further supports the use of CD63 as a general biomarker for detecting and/or monitoring LSDs.

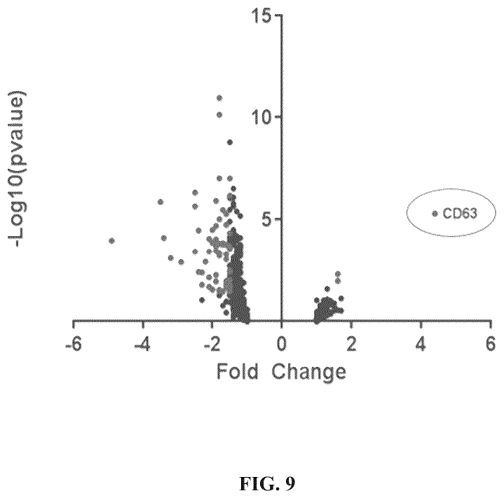

Olink® profiling of the samples from the male adult Fabry patients indicated that CD63 is the protein which showed the most significant upregulation as compared to healthy controls (FIG. 9).

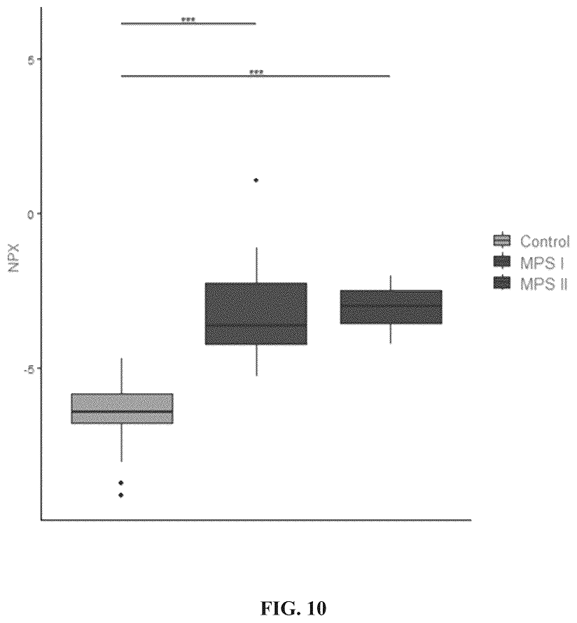

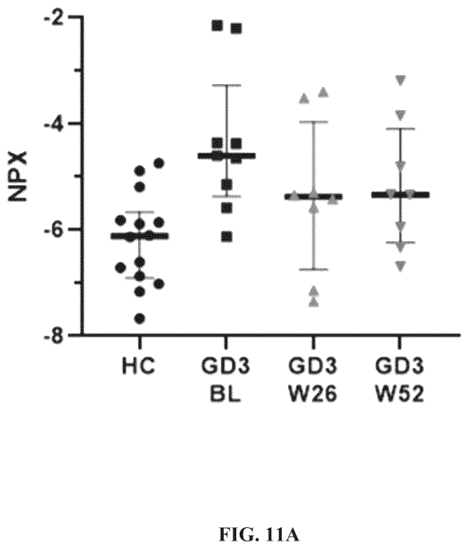

Analysis of the data collected from the MPS CSF samples revealed compelling results in the case of CD63. FIG. 10 (box plot) shows a substantial elevation in CD63 levels (NPX value=−3.186 and −3.037 for MPS I and MPS II, respectively) among MPS patients as compared to the control group (NPX value=−6.402). The p value of differences between MPS I/MPS II and healthy references are all <0.001. These findings strongly support the feasibility and clinical utility of CD63 as a biomarker for MPS in CSF.

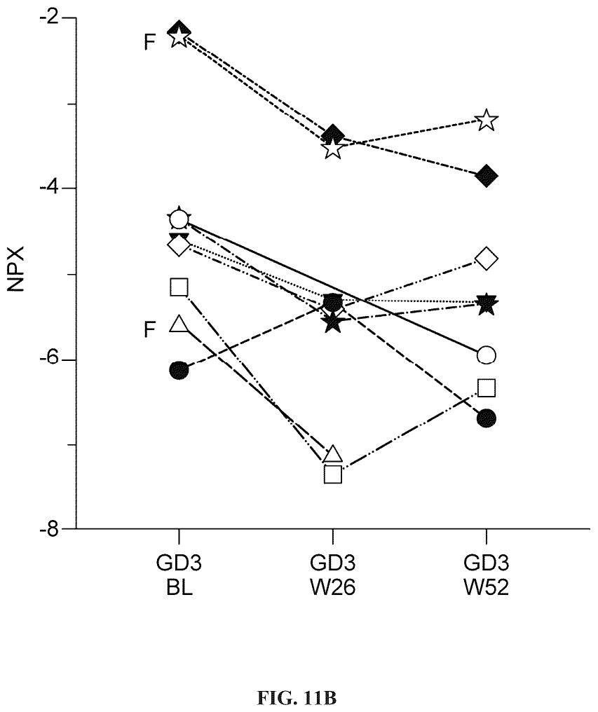

The longitudinal GD3 samples also indicated a significant elevation in CD63 levels in the CSF at baseline as compared to the average in healthy controls (FIG. 11A), as well as a reduction over time on treatment. The same trend was observed in individual patients (FIG. 11B). In particular, there was a substantial elevation of CD63 levels in baseline GD3 patients compared to healthy controls (FIG. 11A), with NPX values of −6.1 and −4.3 for controls and GD3 patients, respectively. Even without considering disease severity, the statistical analysis showed high significance (p value of 0.0036), indicating a clear distinction between the two groups. Furthermore, treatment with venglustat, an investigational glucosylceramide synthase inhibitor that penetrates the brain, consistently led to a decrease in CD63 levels, indicating the effectiveness of the treatment. Moreover, the changes in CD63 showed a positive correlation with the data with the lysoGL-1 change in CSF, further supporting the potential of CD63 as a diagnostic marker for GD3 in CSF.

These findings highlight the potential utility of CD63 as a CSF biomarker for the detection and monitoring of GD3. By evaluating CD63 levels in CSF, we can gain valuable insights into GD3 disease progression and treatment response.

The results discussed above indicate that CD63 is a circulating biomarker useful for assessing lysosomal storage disease pathogenesis in a general sense, especially for LSDs involving the lysosomal glycosphingolipid pathway (for which no single biomarker is currently available). The ready availability of simple assays for quantifying and monitoring CD63, such as ELISAs, along with its proven use in blood samples makes CD63 a particularly attractive biomarker in this context.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1: The method of claim 98, wherein the assessing comprises diagnosing the subject as suffering from, or being at risk of suffering from, a lysosomal storage disease (LSD) when an aberrant level of CD63 is measured in the sample taken from the subject.

2-3. (canceled)

4: The method of claim 98, wherein the assessing comprises detecting or diagnosing aberrant glycosphingolipid processing in the subject when an aberrant level of CD63 is measured in the sample taken from the subject.

5-9. (canceled)

10: The method of claim 98, wherein the assessing comprises:
(i) diagnosing Fabry disease when the aberrant level of CD63 is measured in a subject suspected as being at risk of suffering from Fabry disease;
(ii) diagnosing Gaucher disease when the aberrant level of CD63 is measured in a subject suspected as being at risk of suffering from Gaucher disease; or
(iii) diagnosing mucopolysaccharidosis (MPS) when the aberrant level of CD63 is measured in a subject suspected as being at risk of suffering from MPS.

11-17. (canceled)

18: A method of treating Fabry disease, Gaucher disease, or mucopolysaccharidosis (MPS) in a subject in need thereof, wherein the method comprises:
(i) identifying that the subject has a higher than normal blood plasma CD63 level; and
(ii) treating the subject for Fabry disease, Gaucher disease, or MPS, wherein treating the subject for Fabry disease, Gaucher disease, or MPS comprises administering to the subject an effective amount of a therapeutic treatment for Fabry disease, Gaucher disease, or MPS.

19-24. (canceled)

25: The method of claim 98, wherein the assessing comprises monitoring the progress of Fabry disease, Gaucher disease, or mucopolysaccharidosis (MPS) in a subject diagnosed as having Fabry disease, Gaucher disease, or MPS, the monitoring comprising:
(a) measuring a level of CD63 in a first sample taken from the subject;
(b) measuring a level of CD63 in a second sample taken from the subject, wherein the second sample was taken from the subject after the first sample was taken from the subject;
(c) comparing the level of CD63 in the first sample with the level of CD63 in the second sample; and
(d) determining that the Fabry disease, Gaucher disease, or mucopolysaccharidosis (MPS) in the subject is becoming more severe if the level of CD63 in the second sample is greater than the level of CD63 in the first sample, determining that the Fabry disease, Gaucher disease, or MPS in the subject is not progressing if the level of CD63 in the second sample is substantially the same as the level of CD63 in the first sample, and determining that the Fabry disease, Gaucher disease, or MPS in the subject is becoming less severe if the level of CD63 in the second sample is lower than the level of CD63 in the first sample.

26-27. (canceled)

28: The method of claim 98, wherein the assessing comprises monitoring the progress of a therapeutic treatment for Fabry disease, Gaucher disease, or mucopolysaccharidosis (MPS) in a subject diagnosed as having Fabry disease, Gaucher disease, or MPS, the method comprising:
(a) measuring a level of CD63 in a first sample taken from the subject;
(b) administering a therapeutic treatment for the Fabry disease, Gaucher disease, or MPS to the subject;
(c) measuring a level of CD63 in a second sample taken from the subject, wherein the second sample is taken from the subject after the therapeutic treatment was administered, the second sample being the same type of sample as the first sample; and
(d) determining that the therapeutic treatment is successful if the level of CD63 in the second sample is lower than the level of CD63 in the first sample.

29: A method of treating or preventing the development or progression of Fabry disease, Gaucher disease, or mucopolysaccharidosis (MPS) in a subject assessed as being at risk of suffering from Fabry disease, Gaucher disease, or MPS, the method comprising the steps of:
(a) taking a first biological sample from the subject and analyzing the sample for CD63 concentration;
(b) starting a course of therapeutic treatment for Fabry disease, Gaucher disease, or MPS if the CD63 concentration is above a control value;
(c) after starting the course of therapeutic treatment, taking a second biological sample from the subject and analyzing the sample for CD63 concentration to determine a change in CD63 level; and
(d) administering the therapeutic treatment adjusted based on the change in CD63 level observed.

30: A method for adjusting a dosage of a therapeutic treatment for Fabry disease, Gaucher disease, or MPS in a subject receiving the therapeutic treatment, the method comprising:
(a) measuring the level of CD63 in a first biological sample taken from the subject;
(b) administering one or more doses of the therapeutic treatment to the subject;
(c) measuring the level of CD63 in a second biological sample taken from the subject; and
(d) administering the therapeutic treatment adjusted based on the difference between the level of CD63 in the first biological sample and the level of CD63 in the second biological sample.

31-97. (canceled)

98: A method of assessing lysosomal dysfunction in a subject, the method comprising measuring an aberrant level of CD63 in a sample taken from the subject thereby to assess lysosomal dysfunction in the subject, wherein CD63 is used as a stand-alone biomarker to assess lysosomal dysfunction.

99: The method of claim 98, wherein an aberrant level of CD63 is detected when the level of CD63 in the sample taken from the subject is greater than a control value, wherein the control value is measured as a level of CD63 in a sample taken either from the same subject at an earlier point in time or from one or more healthy subjects.
